(19) United States
(12) Patent Application Publication
(10) Pub. No.: US 2006/0282154 A1
(43) Pub. Date: Dec. 14, 2006

(54) Bifurcation stent delivery catheter assembly and method

(75) Inventors: Randolf Von Oepen, Los Altos Hills, CA (US); Thomas Rieth, Hirrlingen (DE); Lorcan Coffey, Tubingen (DE); Arik Zucker, Zurich (CH); Travis Yribarren, San Mateo, CA (US)

Correspondence Address: BEYER WEAVER & THOMAS LLP, P.O. BOX 70250, OAKLAND, CA, 94612-0250, US

(73) Assignee: Abbott Laboratories, Redwood City, CA (US)

(21) Appl. No.: 11/441,388

(22) Filed: May 24, 2006

Related U.S. Application Data
(60) Provisional application No. 60/684,613, filed on May 24, 2005.
(60) Provisional application No. 60/736,637, filed on Nov. 15, 2005.

(51) Int. Cl. A61F 2/06

(52) U.S. Cl. 623/1.11

(57) ABSTRACT

A stent delivery system is disclosed for delivering and deploying a radially expandable stent at a strategic orientation and location in a body vessel. The delivery system includes an elongated flexible tubular shaft sized suitably for insertion into the body vessel. A stent deployment assembly includes a distal transition portion supporting a dilator device adapted for radial expansion about a longitudinal axis of the deployment assembly from a non-expanded condition to a radially expanded condition. The dilator device is configured to support the stent thereon in the non-expanded condition and in predetermined orientation relative the deployment assembly. A rotational clutch assembly rotatably mounts the transition portion to a distal portion of the tubular shaft such that the deployment assembly is substantially torsionally isolated from the tubular shaft, about a longitudinal axis of the clutch assembly. This enables the stent deployment assembly to rotate substantially independently of the tubular shaft for strategic orientation of the dilator device during advancement through the body vessel.

### RELATED APPLICATION DATA

The present application claims priority under 35 U.S.C. §119 to U.S. Provisional Application Ser. No. 60/684,613, naming Von Oepen et al as inventors, filed May 24, 2005, and entitled BIFURCATION STENT DELIVERY CATHETER ASSEMBLY AND METHOD, and U.S. Provisional Application Ser. No. 60/736,637, naming Von Oepen et al as the inventors, filed Nov. 15, 2005, and entitled the same, both of which are incorporated herein by reference in their entirety and for all purposes.

### FIELD OF THE INVENTION

The present invention relates generally to catheters and systems used for delivering devices such as, but not limited to, intravascular stents and therapeutic agents to sites within vascular or tubular channel systems of the body. More particularly, it relates to delivery catheters and systems for delivering stents to bifurcated vessels.

### BACKGROUND OF THE INVENTION

A type of endoprosthesis device, commonly referred to as a stent, may be placed or implanted within a vein, artery or other tubular body organ for treating occlusions, stenoses, aneurysms or dissections of a vessel by reinforcing the wall of the vessel or by expanding the vessel. Stents are normally placed to scaffold the vessel and avoid elastic recoil after angioplasty. Another reason for applying a stent is it to treat dissections in blood vessel walls caused by balloon angioplasty of the coronary arteries as well as peripheral arteries and to improve angioplasty results by preventing elastic recoil and remodeling of the vessel wall. Two randomized multicenter trials have shown a lower restenosis rate in stent treated coronary arteries compared with balloon angioplasty alone (Serruys, P W et al. New England Journal of Medicine 331: 489-495, 1994, Fischman, D L et al. New England Journal of Medicine 331:496-501, 1994). Stents have been successfully implanted in the urinary tract, the bile duct, the esophagus and the tracheo-bronchial tree to reinforce those body organs, as well as implanted into the neurovascular, peripheral vascular, coronary, cardiac, and renal systems, among others. The term “stent” as used in this Application is a device that is intraluminally implanted within bodily vessels to reinforce collapsing, dissected, partially occluded, weakened, diseased or abnormally dilated or small segments of a vessel wall.

One common procedure for intraluminally implanting a stent within a body vessel is to first dilate the relevant region of the vessel with a balloon catheter. Subsequently, a delivery catheter, such as Percutaneous Transluminal Coronary Angioplasty (PTCA) Catheters containing a dilator at the distal end thereof, is applied to transport a stent to the lesion site, and to deploy the stent in a position that bridges the affected portion of the vessel. The expanded stent provides scaffolding to the lumen that allows adequate blood flow within the lumen. These delivery catheters typically include a relatively long flexible shaft (e.g., normally about 145 cm in length that is sized to be percutaneously inserted into the vessels) with a dilator or stent deployment assembly at the distal end of the shaft that carries the stent.

During any such catheterization and interventional procedures, including for example angioplasty and/or stenting, a hollow needle is initially applied through a patient's skin and tissue to facilitate advancement of the catheter shaft through the target vasculature. As is often the case, however, the catheter shaft may need to be inserted into vessels having a relatively tortuous path leading to the lesion site. Since it can be difficult to steer many types of catheters, guidewires are applied to facilitate advancement of the catheters through the vessel. Guidewires are typically formed from a very small diameter metallic wire having a flexible tip that can be rotatably controlled to some degree. The operator shapes the tip of the guidewire by bending it depending on the anatomy of the vessel. Since the guidewire body is transmitting torque very well, the tip of the catheter can be steered through the anatomy of the patient. Furthermore, steerable guidewires have been developed which allow the operator to deflect the tip of the wire actively in the vasculature of the patient. The ability to rotatably control the tip is important in that the guidewire can be steered to access a desired location through a potentially tortuous path such as the vasculature.

Once the guidewire is advanced through the needle and into the patient's blood vessel, the needle is removed. An introducer sheath is then advanced over the guidewire into the vessel, e.g., in conjunction with or subsequent to a dilator. The catheter or other deployment device may then be advanced through a lumen of the introducer sheath and over the guidewire into a position for performing a medical procedure. Thus, the introducer sheath may facilitate introducing various devices into the vessel, while minimizing trauma to the vessel wall and/or minimizing blood loss during a procedure.

In some applications, the targeted region of a vessel may be at a location where the vessel bifurcates. For example, in cases where plaque has developed in the region of a vessel bifurcation, it may be desirable to perform angioplasty, atherectomy, and/or stenting in one or all of the affected vessels. In general, it is very important to preserve the side branch and the main branch of the bifurcation. In some occlusions, it might occur that during the dilation, plaque will be shifted from the treated vessel to the non-treated vessel, and will then occlude that non-treated vessel. This effect is known as the “snowplow” effect. To enable physicians to reaccess the vessel that has been affected by the “snowplow” effect, most physicians prefer to place a guidewire in the non-treated branch as well. If the non-treated vessel is occluded during this procedure, the guidewire positioned in the non-treated vessel will function as a guiding element, and will allow the advance of another catheter to reopen that vessel. In other applications, it may be desirable to insert a bifurcation stent specifically dedicated to treat lesions at a vessel bifurcation.

In the recent past, several commercially available bifurcation stents have been developed that treat bifurcation lesions. By way of example, common alternatives to bifurcation lesion stenting include the Elective T technique, the Provisional T Technique, the Coulotte Technique, the V Technique and the Crush. In addition, dedicated bifurcation systems like the Frontier and AST Systems have been developed. While these bifurcation stent designs have encountered varying degrees of success, one major problem associated with all bifurcation systems is that the delivery and deployment of the stent, relative to the side branch, is extremely difficult. This is due primarily to the difficulty in properly controlling the orientation, alignment and position of the stent deployment assembly relative to the main branch and side branch of the bifurcated vessel.

During advancement of the catheter shaft along the predisposed guidewire, the stent deployment assembly, which supports and transports the stent in a collapsed state, is not rotatably controlled. Hence, it is likely necessary to rotate and reorient the distal delivery assembly about its longitudinal axis since the bifurcation stent must be properly aligned relative to the side branch before deployment. Transmitting a controlled rotation to the distal end of the catheter over the length of the flexible catheter shaft, however, is nearly impossible. Due in part to the complex anatomy of a coronary artery, the flexible catheter shaft will not adequately transfer torque to the dilatory. Although a proximal portion of the delivery catheter, which often includes a relatively rigid material such as a hypotube or a polymeric tube with a stiffening wire, can reasonably transmit torque, the more distal portions of the flexible catheter shaft cannot. Typically, the elongated, flexible catheter shaft will just rotate at the proximal portion without transmitting such rotational displacement to the dilator in a consistent manner.

Accordingly, there is a need for a stent delivery system with improved alignment and orientation capabilities of the distal stent deployment assembly for those stents (e.g., bifurcation stents) that require precise rotational alignment, about their longitudinal axis, relative to the target vessel site.

### SUMMARY OF THE INVENTION

The present invention is directed toward a stent delivery system for delivering and deploying a radially expandable stent at a strategic orientation and location in a body vessel. The delivery system includes an elongated shaft, and a stent deployment assembly including a proximal transition portion associated with a dilator device. The dilator device is adapted for radial expansion from a non-expanded condition to a radially expanded condition, and further configured to retain the stent in the non-expanded condition. A rotational clutch assembly is included that is configured to rotatably mount the transition portion to a distal portion of the elongated shaft such that the deployment assembly is substantially torsionally isolated from the elongated shaft.

Accordingly, when two guidewires are disposed in a main branch and a side branch at a carina of a bifurcated body vessel, the relatively freely rotatable distal stent deployment assembly can be more easily radially aligned about its longitudinal axis (i.e., with less resistance). Consequently, as the elongated shaft is advanced along the guidewires through the body vessel, the stent deployment assembly is self-aligned with the side branch for strategic orientation and deployment of the stent. Moreover, such relatively free rotational displacement of the stent deployment assembly improves the ability to unwind and navigate through twists in the guidewires as the delivery assembly is advanced along the wires.

In one specific embodiment, the clutch assembly is adapted to transmit compression forces longitudinally along the distal portion of the elongated shaft to the deployment assembly during advancement of the elongated shaft through the body vessel, as well as transmit tension forces during retraction of the shaft.

In another arrangement, the clutch assembly includes an inwardly tapered shoulder portion coupled to one of a distal end of the elongated shaft and a proximal end of the transition portion. The clutch assembly further includes a neck portion extending from the tapered shoulder portion. The neck portion is formed and dimensioned for sliding rotational receipt into an opening at the other of the tubular transition portion and the elongated shaft for rotational receipt thereof.

A flexible protective boot device, in another specific embodiment, extends circumferentially over the clutch assembly having one end secured to the elongated shaft and an opposite end secured to the support shaft forming a fluid-tight seal while still enabling relative rotation between the elongated shaft and the deployment device.

In another aspect of the present invention, a first guidewire lumen is included that extends along at least a portion of the stent deployment assembly. The first guidewire lumen is sized and dimensioned for sliding receipt of a first guidewire disposed in the body vessel. A second guidewire lumen or passage further extends along at least a portion of the stent deployment assembly, and terminates strategically along the dilator device of the stent deployment assembly. The second guidewire lumen or passage is sized and dimensioned for sliding receipt of a second guidewire disposed in the body vessel. The second guidewire lumen or passage is offset from the first guidewire lumen such that during advancement along the first and second guidewires, the deployment assembly will be caused to rotate into alignment with the position of the second guidewire relative the first guidewire.

The clutch assembly may include a pair of opposed contact elements disposed in opposed relationship to one another. One contact element is associated with the elongated shaft while the second contact element is associated with the transition portion. During advancement of the elongated shaft through the body vessel, the contact elements are moved into compressive mutual contact with one another to transmit axial compressive forces from the elongated shaft to the transition portion. In one particular embodiment, the clutch assembly includes a first support tube associated with the elongated shaft, and a second support tube associated with the transition portion. Each support tube includes a respective end portion substantially in opposed relationship to one another, and each end portion supporting one of the contact elements in opposed relationship to one another.

An elongated stiffening element may be included that extends substantially longitudinally the clutch assembly. One end of the stiffening element is disposed in a distal pocket defined in part by a distal end wall of the transition portion, and an opposite end of the stiffening element is disposed in a proximal pocket defined in part by a proximal end wall of the elongated shaft. During the advancement of the elongated shaft through the body vessel, one end of the stiffening element contacts the distal end wall and the opposite end of the stiffening element contacts the proximal end wall to transmit axial compressive forces from the elongated shaft to the transition portion.

In yet another embodiment, the clutch assembly includes an outer tubular flexible member having a proximal end associated to the elongated shaft and a distal end associated to the transition portion. The proximal end and the distal end of the flexible member are configured to rotate relatively freely with respect to one another about a longitudinal axis of the flexible member.

The clutch assembly further includes an inner tubular flexible member disposed substantially co-axially within the outer tubular flexible member. A proximal end of the inner flexible member is associated to the proximal tube segment and a distal end is associated to the distal tube segment. The first guidewire passage, thus, extends continuously through the elongated shaft, the clutch assembly and the stent deployment assembly. The proximal end and the distal end of the inner flexible member are configured to rotate relatively freely with respect to one another about the longitudinal axis of the outer flexible member.

In one particular configuration both the outer and inner tubular flexible members are wound structures having a plurality of coils. A respective proximal end coil of the plurality of coils associated with the elongated shaft and proximal tube segment, and a distal end coil of the plurality of coils is associated to the transition portion and the distal tube segment, respectively.

Each wound member may include a fluid impermeable, cylindrical-shaped inner sealing member disposed adjacent to the respective tubular flexible member. A respective proximal end of each sealing member is affixed to the proximal tube segment or elongated shaft in a fluid-tight manner, and a respective distal end thereof is affixed to the distal tube segment or transition portion in a fluid-tight manner to prevent fluid penetration therethrough.

Still another specific embodiment provides a standoff feature disposed between the inner tubular flexible member and the outer tubular flexible member. During a collapse of the outer tubular flexible member onto the inner flexible tubular member under a vacuum, the stand-off feature cooperates with the tubular flexible members to define at least one fluid communication channel extending longitudinally along the clutch assembly from a proximal end to a distal end thereof.

In one embodiment, the standoff feature includes a plurality of longitudinally extending protrusions disposed radially about the inner flexible tubular member. Each protrusion extends radially outward in a direction toward the outer flexible tubular member. The protrusions may be integral with the inner flexible member, but may also be provided by the protective sealing member.

In still another arrangement, the standoff feature includes one or more elongated wound members wound about a respective longitudinal axis. These are disposed between the inner flexible tubular member and the outer flexible tubular member. A respective longitudinal axis of the one or more wound members is offset from the longitudinal axis of the inner tubular flexible member.

In yet another embodiment of the standoff feature, one elongated wound member is provided wound about the inner flexible tubular member. The longitudinal axis of the wound member is substantially co-axial with the longitudinal axis of the inner tubular flexible member.

In another aspect of the present invention, a rotational clutch assembly is provided for a stent delivery catheter for delivering and deploying a radially expandable stent at a strategic orientation and location in a body vessel. The clutch assembly includes a tubular transition portion having a distal end mounted to the dilator device. A proximal portion of the transition portion is rotatably coupled to the distal end of the elongated shaft at rotational joint for substantially free rotation about a longitudinal axis thereof relative to the elongated shaft. Hence, the dilator device of the catheter is substantially torsionally isolated from the elongated shaft. The clutch assembly further includes a pair of opposed contact elements disposed in opposed relationship to one another. One contact element is associated with the elongated shaft with the second contact element being associated with the transition portion. During advancement of the elongated shaft through the body vessel, hence, the contact elements are moved into compressive mutual contact with one another to transmit axial compressive forces from the elongated shaft to the transition portion.

### BRIEF DESCRIPTION OF THE DRAWINGS

The assembly of the present invention has other objects and features of advantage that will be more readily apparent from the following description of the best mode of carrying out the invention and the appended claims, when taken in conjunction with the accompanying drawing, in which:

FIG. 1A is a side elevation view, in cross-section, of a stent delivery system, as constructed in accordance with the present invention.

FIG. 1B is a front elevation view, in cross-section, of a stent deployment assembly of the stent delivery system taken substantially along the plane of the line 1B-1B in FIG. 11A, and illustrating the formation of a second guidewire lumen.

FIG. 2 is an enlarged, side elevation view, in cross-section, of a clutch assembly of the stent delivery system of FIG. 1A.

FIGS. 3 and 4 are a series of side elevation views, in cross-section, of the stent delivery system of FIG. 1A, being advanced through a bifurcated vessel and illustrating use and alignment thereof.

FIG. 5 is a side elevation view, in cross-section, of another specific embodiment stent delivery system, illustrating the opposed support tubes that provide axial stiffness.

FIG. 6 is a side elevation view, in cross-section, of the stent delivery system of FIG. 1A with a dilator device in an expanded condition.

FIG. 7A is a side elevation view, in cross-section, of another specific embodiment stent delivery system, illustrating the opposed support bands mounted to the opposed support tubes that provide axial stiffness.

FIG. 7B is a side elevation view, in cross-section, of another specific embodiment clutch assembly of the stent delivery system, illustrating opposed contacting stoppers mounted to the opposed elongated shaft and the transition portion of the stent delivery assembly.

FIG. 8 is a side elevation view, in cross-section, of another specific embodiment stent delivery system, illustrating the incorporation of a Nitinol wire axial stiffener.

FIG. 9 is a side elevation view, in cross-section, of a bifurcated vessel, and illustrating the typical geometry of two guidewires disposed in the bifurcated vessel.

FIG. 10 is a fragmentary, side elevation view, in cross-section, of the clutch assembly.

FIG. 11 is a fragmentary, side elevation view, in cross-section, of another specific embodiment clutch assembly of the present invention containing a plurality of interlocking tube elements and a bellow-type protective boot.

FIG. 12 is a fragmentary, side elevation view, in cross-section, of an alternative for the interlocking tube element clutch assembly of FIG. 11.

FIG. 13 is an enlarged, side elevation view, of the plurality of interlocking tube elements for the interlocking tube element clutch assembly of FIG. 11.

FIG. 14 is a top plan view of a flatten pattern of a tube structure to fabricate the annular bushing of FIG. 15.

FIG. 15 is a front elevation view of an annular bushing for the interlocking tube element clutch assembly of FIG. 11.

FIG. 16 is a top plan view of a flattened pattern of a tube structure to fabricate the interlocking tube elements for the clutch assembly of FIG. 12.

FIGS. 17A-17E is a sequence of schematic, top perspective views of a dilator device of the stent delivery system of the present invention, illustrating formation of the second guidewire lumen using a mandrel.

FIG. 18 is a schematic, top perspective view of a dilator device of the stent delivery system in accordance with the present invention, illustrating formation of the second guidewire lumen within a fold of the dilator device.

FIG. 19 is a side elevation view, in cross-section, of another specific embodiment stent delivery system illustrating positioning of a distal segment of the second guidewire tube along the stent deployment assembly.

FIG. 20 is a side elevation view, in cross-section, of another specific embodiment stent delivery system illustrating mounting of the first and second guidewire tubes to an exterior of the tubular shaft.

FIG. 21 is a fragmentary, side elevation view, in cross-section, of another specific embodiment stent delivery system showing passage of the two guidewire lumens internally through the clutch assembly.

FIG. 22 is a fragmentary, side elevation view, in cross-section, of another specific embodiment stent delivery system incorporating a long arm catheter.

FIG. 23 is a side elevation view, in cross-section, of another specific embodiment stent delivery system, incorporating an outer protective boot and a central stiffening wire.

FIGS. 24 and 25 are fragmentary, side elevation views, in cross-section, of another specific embodiment stent delivery system incorporating a double arm catheter.

FIG. 26 is a side elevation view, in cross-section, of another specific embodiment stent delivery system illustrating the application of two clutch assemblies.

FIG. 27 is a side elevation view, in cross-section, of another specific embodiment stent delivery system incorporating a torque-transmitting device.

FIG. 28 is a side elevation view, in cross-section, of another specific embodiment stent delivery system incorporating another specific embodiment torque-transmitting device.

FIG. 29 is a partial cross-sectional view of another specific embodiment of a clutch in accordance with the present invention.

FIGS. 30A to 30D are cross-sectional views of features that may be formed in the surface of the coating or sleeve disposed about the inner flexible member.

FIG. 31 is a partial cross-sectional view of one specific embodiment of a clutch assembly in accordance with the present invention further including an additional inner flexible member.

FIG. 32 is a partial cross-sectional view of one specific embodiment of a clutch assembly in accordance with the present invention further including an additional flexible member.

FIG. 33 is a fragmentary, top perspective view of another specific embodiment of the clutch assembly for the stent delivery system of FIG. 29, axially staggering in position of the inner and outer clutch assemblies.

FIG. 34 is a fragmentary, side elevation view of the clutch assembly embodiment of FIG. 33.

FIG. 35 is an enlarged, fragmentary, side elevation view, in partial cross-section, of the outer clutch assembly of FIG. 34.

FIG. 36 is an enlarged, fragmentary, side elevation view, in partial cross-section, of the inner clutch assembly of FIG. 34.

FIG. 37 is a fragmentary, side elevation view, in cross-section, of the outer clutch assembly of FIG. 35.

FIG. 38 is a fragmentary, side elevation view, in cross-section, of the inner clutch assembly of FIG. 36.

### DETAILED DESCRIPTION OF THE PREFERRED EMBODIMENTS

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

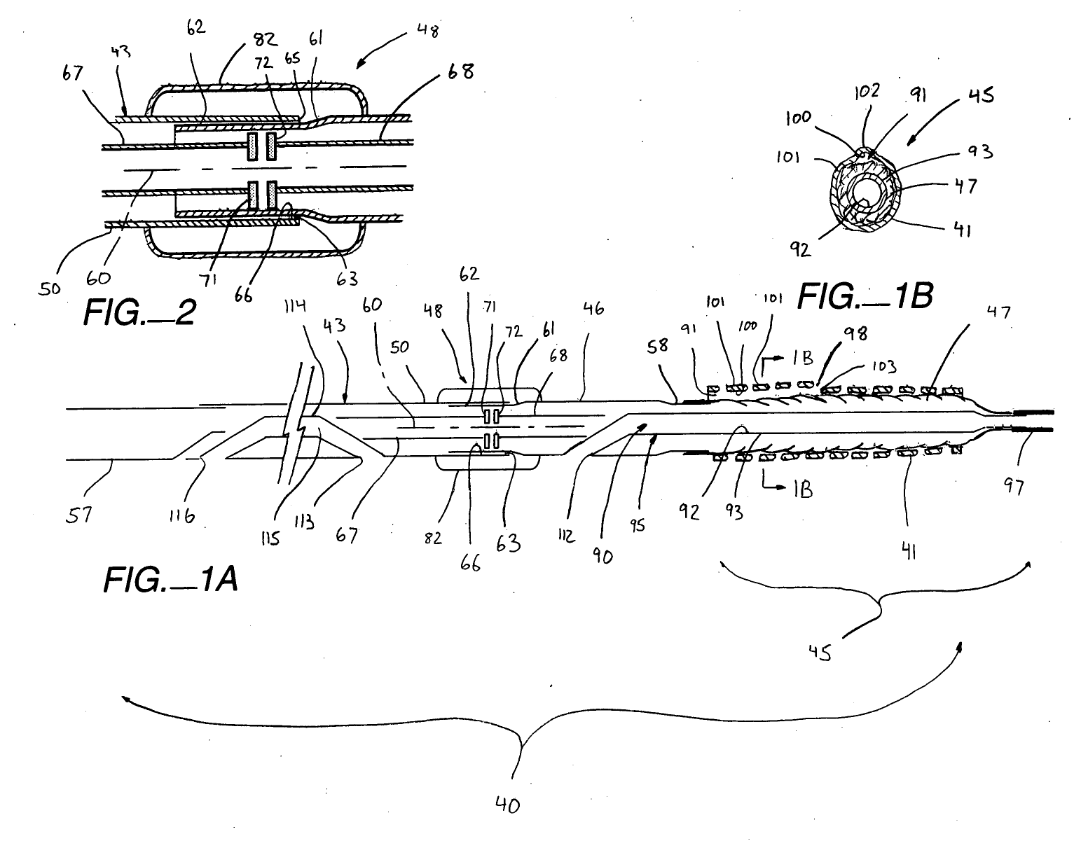

Referring now to FIGS. 1-5, a stent delivery system, generally designated 40, is described that delivers and deploys a radially expandable stent 41 (e.g., a bifurcation stent) at a strategic orientation and location in a body vessel 42. The delivery system 40 includes an elongated shaft 43 sized suitably for insertion into the body vessel 42. A stent deployment assembly 45 includes a tubular distal transition portion 46 supporting a dilator device 47 adapted for radial expansion about a longitudinal axis of the deployment assembly 45 from a non-expanded condition (FIG. 1, 3-4) to a radially expanded condition (FIGS. 5-8). The dilator device 47 is configured to support the stent 41 thereon in the non-expanded condition, and in predetermined orientation relative to the deployment assembly 45. The stent delivery system 40 includes a joint device or clutch assembly 48 configured to rotatably mount the transition portion 46 to a distal portion 50 of the proximal elongated shaft 43 such that the stent deployment assembly 45 is substantially torsionally isolated from the elongated shaft. This allows the dilator device 47 to rotate substantially independently from elongated shaft 43 for strategic orientation of the dilator device during advancement through the body vessel.

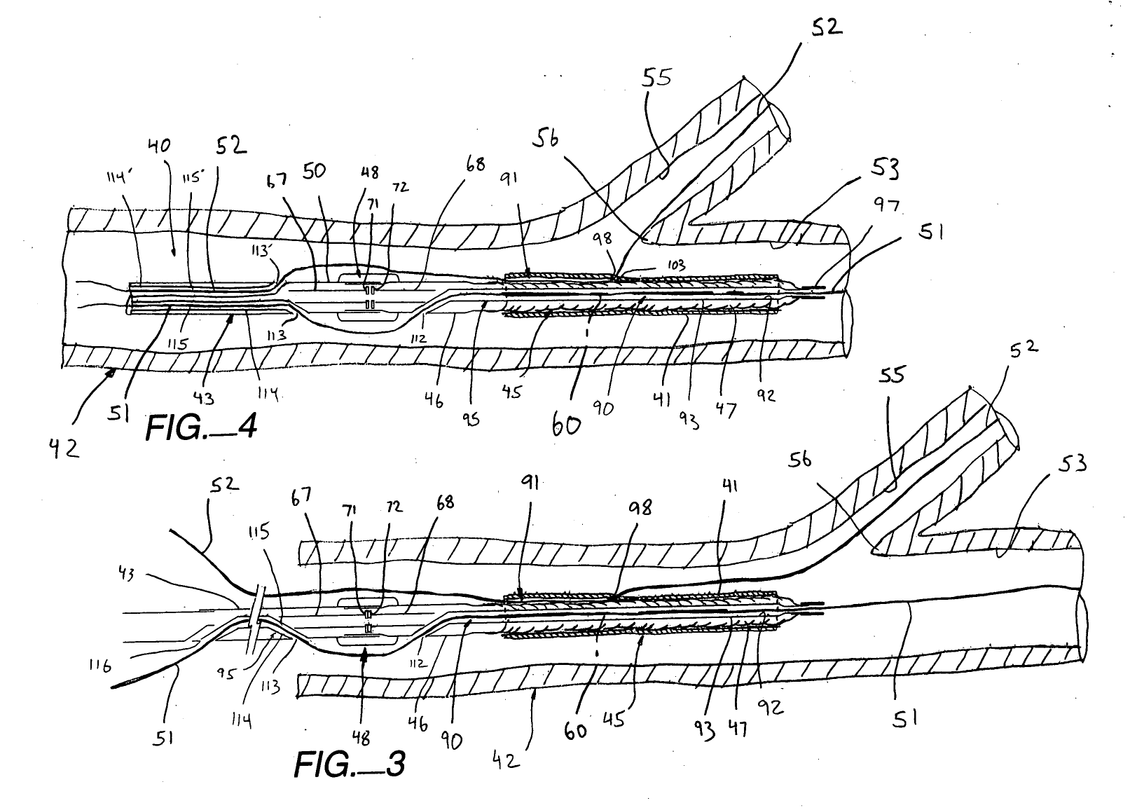

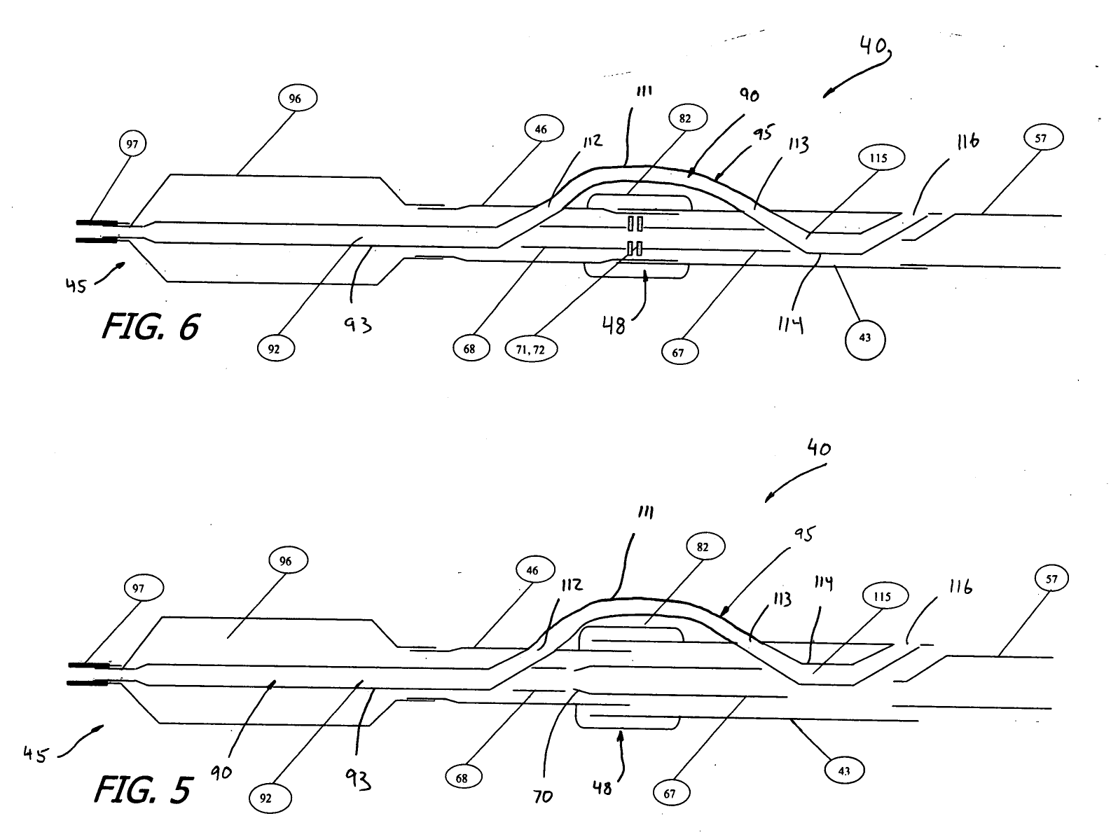

Accordingly, using a conventional two guidewire delivery system (FIGS. 3, 4 and 28), the distal stent deployment assembly 45 (carrying the strategically aligned stent) cooperates with the two guidewires 51, 52 for rotational alignment thereof, about their longitudinal axis 60, as the catheter system is advanced through the vessel along the guidewires. Briefly, one of the guidewires is disposed in a main branch 53 of the body vessel 42, while the other guidewire is disposed in a side branch 55 at a carina 56 using conventional deployment techniques such as those described in WO 01/30433 A1, published May 3, 2001, entitled “A GUIDEWIRE POSITIONING DEVICE”, and herein incorporated by reference in its entirety. Applying the two guidewires 51, 52 as an alignment vehicle, the relatively freely rotatable distal stent deployment assembly can be more easily radially aligned about its longitudinal axis (i.e., with less resistance) than in current delivery systems that would require rotation or torquing of both the stent deployment assembly 45 and the elongated shaft 43. Consequently, as the elongated shaft 43 is advanced along the guidewires 51, 52 through the body vessel, the stent deployment assembly 45 is self-aligned with the side branch 55 (as will be described in greater detail below in reference to FIG. 4) for strategic orientation and deployment of the stent. Moreover, such relatively free rotational displacement of the stent deployment assembly 45 improves the ability to unwind and navigate through twists in the guidewires as the delivery assembly is advanced along the wires.

A certain amount of wire crossing or wrapping cannot be avoided. For example, as shown in FIG. 9, at a vessel carina 56, the wires 51, 52 will align according to the vessel geometry. Based upon the wire tension and the position of the main branch 53 and diverging side branch 52, at the very least, two wires crossing occur that many catheter systems are often not capable of following the required rotation to navigate through. Hence, it is highly beneficial if the wire twisting is limited to less than about 360°. Especially when the system is advanced in the carina, then it has to be assured that the delivery system will allow the stent to be correctly aligned with the geometry of the bifurcation. However, it is conceivable that all clutch embodiments of the present invention are capable of at least three full rotations in either direction to negotiate twists and unwinding of the guidewires during advancement through a vessel. In fact, the rotation of the clutch assembly may only be limited by the elasticity of the protective boot or sleeve covering the same (e.g. boot 82 as will be described).

Referring back to FIG. 1, the stent delivery system 40 may primarily be provided by a conventional balloon catheter apparatus with an elongated flexible tubular shaft 43 composed of any conventional catheter material. Such materials, by way of example, include stainless steel, nylon, PTFE, Pebax, and carbon fiber. The shaft length is typically in the range of about 60 cm to about 300 cm, but may vary of course depending upon the application. Further, the shaft diameter is typically in the range of about 0.3 mm to about 7.0 mm, which is suitable for insertion into most vessels of the coronary artery. Other dimensions may be more suitable to use within other vascular or tubular systems of the human body. Such a diameter is also suitable to accommodate one or more access lumens within the tubular shaft such as an inflation or fluid supply lumen, as well as one or more guidewire lumens.

On the proximal end of the tubular shaft 43 is an adapter 57 mainly used for inflation/deflation. Preferably, the adapter 57 is elongated and suitable for gripping and manual support, manipulation and operation of the delivery system and its components thereof.

While the clutch assembly 48 is primarily illustrated as positioned at the distal end of the tubular shaft 43, it may be positioned more proximally along the shaft 43 wherein the transition portion 46 is actually a more distal section of the tubular shaft 43. In general, as shown, however, the stent deployment assembly 45 is rotatably mounted to the distal end of the tubular shaft 43, via clutch assembly 48. FIGS. 1 and 2 illustrate that the stent deployment assembly 45 includes the tubular transition portion 46 having a proximal end coupled to the clutch assembly 48 and a distal end supporting the dilator device 47. The transition portion, which is preferably straight and relatively short, provides axial support to the dilator device 47 during advancement through the body vessel. Further, the diameter of the transition portion 46 is substantially the same as that of the proximal tubular shaft 43.

A distal portion of the transition portion 46 can be tapered inwardly to form a nipple portion 58 that accommodates mounting of the dilator device 47 thereon. In other arrangements, the transition portion can be part of the dilator device. For example, the transition device might be dilated to allow the dilator to be placed inside before connecting the components. In other instances, the dilator device may be mounted using an angled weld, an abutting weld or using inner and/or outer reinforcement tubes. The tubular transition portion 46 may also include or act as part of an inflation lumen that communicates with the dilator device 47 for inflation thereof.

This dilator device 47 may be provided by any conventional system capable of selective radial expansion about a longitudinal axis of the delivery assembly 45 between a non-expanded condition (FIGS. 1, 3 and 4) and the expanded condition (FIGS. 5-7). During advancement of the stent deployment assembly 45 through the body vessel 42, the dilator device 47 is of course maintained in a substantially non-expanded condition with the stent (not shown) strategically mounted thereon in a collapsed or crimped state.

The dilator device 47 can be provided by one of many radially expandable delivery devices. In particular, however, the dilator device is an expanding member-type device or the like that causes selective expansion of its elements, such as a balloon, an expandable mesh or a slit hypotube, etc.

As mentioned, a clutch assembly 48 is disposed between the tubular shaft 43 and the stent deployment assembly 45 near the distal portion of the stent delivery system 40. The clutch assembly 48 is designed to provide independent, relatively resistance-free rotational displacement of the stent deployment assembly 45 generally about a longitudinal axis 60 of the clutch assembly, in relation to the proximal portion of the stent delivery system 40. In accordance with the present invention, however, the clutch assembly 48, as mentioned, must also be capable of transmitting axial compression forces from the tubular shaft 43 to the stent deployment assembly 45, as well as transmitting tension forces. Such compression force transmission by the clutch assembly 48 is necessary to enable advancement of the stent deployment assembly through the body vessel 42. Accordingly, the clutch assembly 48 functions as a dampener between the tubular shaft 43 and the stent deployment assembly 45 such that the torsion forces inflicted upon stent deployment assembly by the two guidewires during vessel advancement are not further resisted by the tubular shaft 43. Hence, the stent deployment assembly can more easily rotate about the longitudinal axis 60 to accommodate the position of the guidewires (as will be explained in greater detail below) since it is rotationally isolated, via the clutch assembly 48, from torsion resistance generated by the tubular shaft 43. Further, the clutch assembly simultaneously transmits the axial compressive forces from the tubular shaft 43 to the stent deployment assembly 45 during said advancement and resists buckling due to resistive forces imparted by the body vessel 42 during advancement.

As best shown in FIG. 2, the rotational joint device or clutch assembly 48 is provided by “slip-fit” a male and corresponding female component arrangement that provides both rotational displacement about the longitudinal axis 60, as well as providing axial compression force transmission. In one specific embodiment, the proximal end of the transition portion 46 includes an inwardly tapered shoulder portion 61 that intersects a neck portion 62 that further extends proximally along the longitudinal axis 60 thereof. The neck portion 62 is substantially cylindrical or may slightly taper inwardly. The diameter of the neck portion is smaller than that of the distal end of the tubular shaft by the amount substantially equal to the tapered shoulder portion 61.

The corresponding female component of the clutch assembly 48 is provided by a receiving socket 63 formed at the distal end of the tubular shaft 43. This distal receiving socket is sized for sliding slip-fit of the neck portion 62 in a manner provided substantially resistance-free rotation of the transition portion 46 about the longitudinal axis 60 of the clutch assembly 48. FIG. 2 best illustrates that the distal end portion of the tubular shaft 43 includes a proximal rim portion 65 that defines an opening into the receiving socket 63. The receiving socket 63 of the clutch assembly 48 is further defined by a substantially cylindrical interior wall 66 having a diameter slightly larger than that of the mating neck portion 62 of the clutch assembly.

The diametric tolerance between the interior walls 66 of the receiving socket 63 and the cylindrical exterior surface of the neck portion 62 is sufficient to enable substantially resistance-free rotational displacement of the neck portion in the receiving socket 63, while at the same time providing sufficient lateral support should such support be required during catheter advancement through the vessel. To reduce friction between the contacting components, they may be coated with PTFE (i.e., TEFLON®) or include other types of lubricants, coatings, or lubricious materials. Such biocompatible lubricants and/or materials are well known in the field and included herein.

In one specific embodiment, the clutch assembly 48 coaxially aligns the stent deployment assembly 45 with tubular shaft 43. Upon longitudinal receipt of the neck portion 62 in the receiving socket 63, hence, the longitudinal axis of the stent deployment assembly 45 is substantially coaxial with the longitudinal axis 60 of the clutch assembly 48, and with that of the distal portion 50 of the tubular shaft 43. Such axial alignment is preferable to retain the small overall diametric footprint at the distal portion of the delivery system 40.

The clutch assembly 48 further includes a pair of opposed support tubes 67, 68 that provide axial stiffening and stability, and to transmit the axial compression forces during contact therebetween when the delivery system is in a compressive state. As best shown in FIGS. 1-6, a proximal support tube 67 is disposed and supported within the distal portion 50 of the tubular shaft 43, and is generally positioned along the longitudinal axis 60 of the clutch assembly. Similarly, an opposed distal support tube 68 is disposed and supported within the proximal portion of the distal transition portion 46, and is generally positioned along the longitudinal axis 60 of the clutch assembly as well. When the clutch assembly 48 is assembled where the neck portion 62 is slip-fit into the receiving socket 63 of the tubular shaft 43, the distal end of the proximal support tube 67 is also slideably received through a proximal opening into the tubular transition portion 46 and into opposed co-axial relationship with the proximal end of the distal support tube 68. Hence, during compression, such as during advancement of the tubular shaft 43 through the body vessel as shown in FIG. 3, the proximal support tube 67 axially contacts the distal support tube 68. Such axial contact provides axial stiffening and stability, and transmits the axial compressive forces urged upon the tubular shaft 43 to the stent deployment assembly 45. However, when the clutch assembly 48 is not in a compressive state, such as shown in FIGS. 2 and 4, the opposed tubes 67, 68 are not in sufficient axial contact, permitting the clutch assembly to rotate about its longitudinal axis 60. The clutch assembly can also rotate when the opposed tubes are in contact.

In one specific embodiment, as shown in FIG. 5, the distal end portion 70 of the proximal support tube 67 tapers radially inward. When the clutch assembly is in a compressed state, this conical-shaped distal end portion 70 contacts the proximal end of the distal support tube 68, providing axial stiffness. Likewise, the proximal end of the distal support tube 68 may be tapered to permit contact with the distal end of proximal support tube 67. In another configurations, as illustrated in FIGS. 1-4 and 6, a pair of contact washers 71, 72 or the like are fixedly disposed on the distal and proximal ends of the opposed support tubes 67, 68. When the clutch assembly 48 is in a compressive state, the opposed contact washers 71, 72 axially contact one another to provide axial stiffness, and enable the transmission of axial compressive forces from the proximal support tube 67 to the distal support tube 68 (FIG. 3).

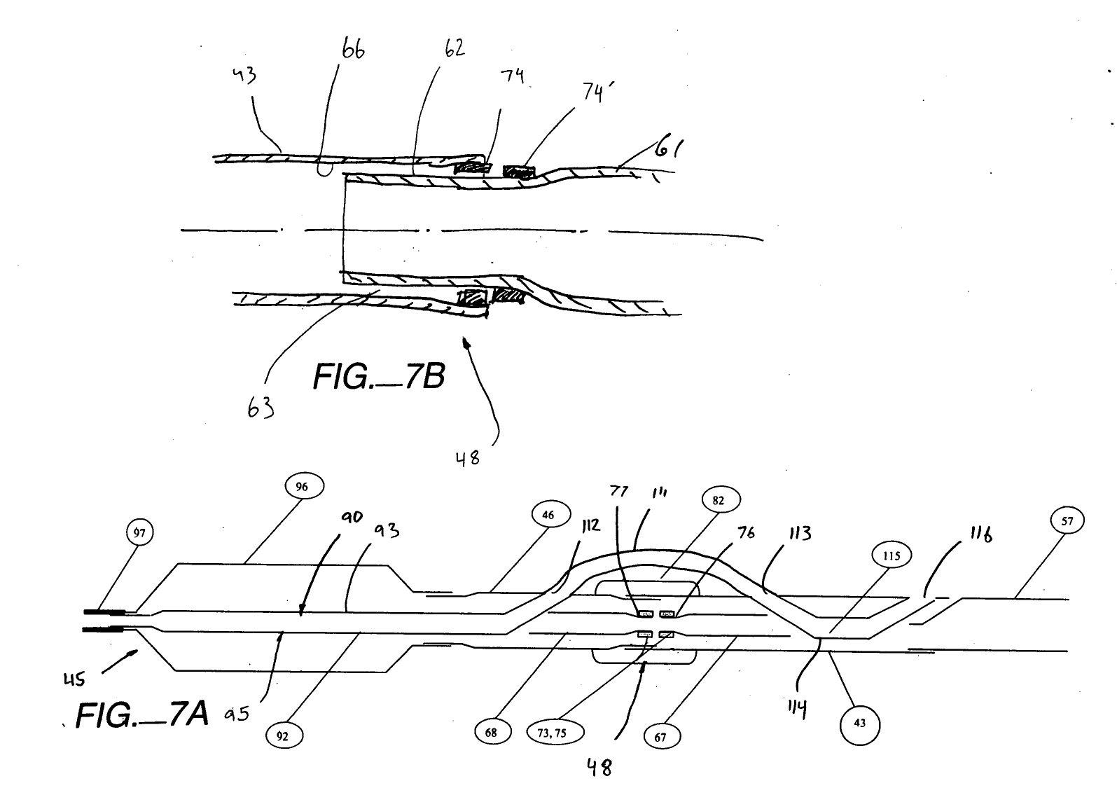

In another specific embodiment, a set of bands 73, 75 are provided around the opposed neck down portions 76, 77 of the corresponding support tubes 67, 68 (FIG. 7A). Similar to the contact washers, these bands 73, 75 contact one another during a compressed state of the clutch assembly 48 to provide axial stiffness therebetween. Such bands 73, 75 can be wholly or partially formed from a radiopaque material to facilitate observation and positioning of the clutch assembly 48 using fluoroscopy or other imaging systems. These support bands 73, 75 can be composed of any suitable material, and even be made from cured adhesive. These bands 73, 75 can be crimped, glued, swaged etc. to the support tubes.

Alternatively, as shown in FIG. 7B, a pair of contact or stoppers 74, 74′ may be incorporated about the neck portion 62, and along the interior wall 66 of the receiving socket 63. These stoppers 74, 74′ are preferably metallic (e.g., RO Markers). The outer shaft 43 may be enlarged and shrinked over the metallic stopper 74, or the metallic stopper will be attached on the inside. A similar procedure will be applied to attach the other metallic stopper 74′.

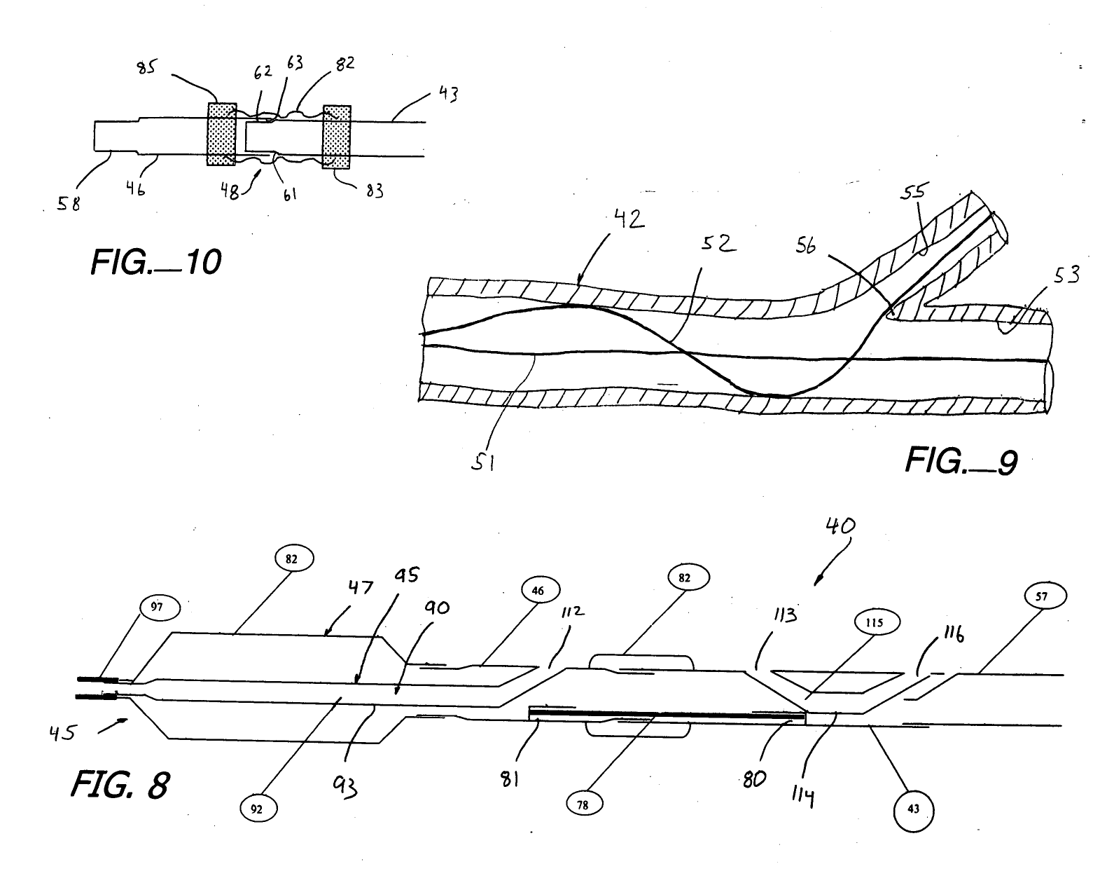

In still another specific embodiment that promotes axial stiffness, as shown in FIG. 8, an elongated wire 78 is disposed in a set of pockets 80, 81 formed between the distal portion 50 of the tubular shaft and the proximal portion of the transition portion 46. These corresponding pockets 80, 81 extend circumferentially about the longitudinal axis of the clutch assembly 48, permitting rotation of the stent deployment assembly 45 relative the tubular shaft. Alternatively, the wire may be positioned central to the clutch assembly.

Upon compression of the clutch assembly 48, the opposed ends of the wire 78 contacts the opposed ends of the pocket 80, 81 to provide axial stiffness. The wire 78 can be fixed at either one or both ends or simply be free-lying within the pockets. The wire may be constructed from Nitinol, or any other metallic material that provides suitable flexibility and mechanical characteristics. Other material exhibiting such characteristics and properties may be utilized such as a carbon rod or the like.

In other configurations, the neck portion 62 and/or the shoulder portion 61 of the clutch assembly 48 may simply abut or contact the interior walls 66 and/or the rim portion 65, respectively, of the transition portion 46. Hence, when an axial driving force is urged upon the tubular shaft 43 during vessel advancement, the contact between the components of the clutch assembly 48 transmit the forces to the stent delivery device to further advance the same through the vessel.

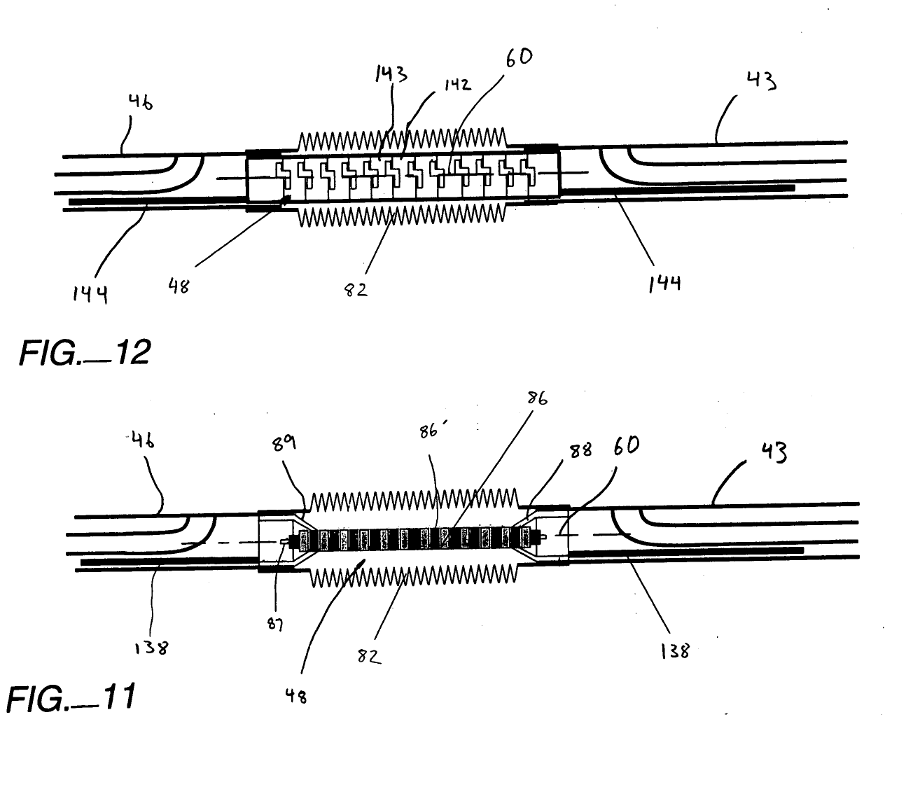

It will be appreciated that while the clutch assembly 48 of the present invention is shown and described in the configurations of FIGS. 1-8 as having the neck portion 62 contained on the proximal end of the distal transition portion 46 and the receiving socket 63 contained on the distal end of the proximal tubular shaft 43, the mating components can be easily reversed without departing from the true spirit and nature of the present invention. For example, as illustrated in FIGS. 10 and 22, the neck portion 62 extends distally from the tubular shaft 43, while the receiving socket 63 is at the proximal end of the distal transition portion 46.

The clutch assembly 48 further includes a cylindrical shell-shaped protective boot 82 or the like to provide a fluid-tight seal around the clutch assembly components (FIGS. 1 and 2). By affixing a proximal portion of the protective boot 82 to the outer or inner circumferential surface of the distal portion 50 of the tubular shaft 43, a fluid tight seal at the proximal end can be formed. Similarly, a distal portion of the protective boot 82 can be affixed to the outer or inner circumferential surface of the transition portion 46, forming another circumferential fluid tight seal. Collectively, the protective boot 82 seals the clutch assembly components therein.

The protective boot may be bonded to the outer circumferential surfaces of the tubular shaft 43 and the transition portion 46 using any biocompatible adhesive or weld material. For example, a transition bonder or any other conventional bonding techniques can be applied such as shrink tubes and hot air, jaw welding, RF welding, UV hardening adhesive, laser welding, white light welding, etc. In another specific embodiment, as shown in FIG. 10, the proximal and distal ends of the protective boot 82 may be embedded in a pair of mounting sleeves 83, 85 that are mounted to the outer circumferential surfaces of the tubular shaft 43 and the transition portion 46.

In accordance with the present invention, since the ends of the protective boot 82 are affixed to the transition portion 46 and the distal portion 50 of the tubular shaft, the rotational displacement at the clutch assembly 48 will be limited to such rotation afforded by the twisting of the boot 82. Therefore, depending upon the size and fitment (e.g., excess looseness) of the protective boot 82, relative the clutch assembly 48, as well as the material properties of the boot, more or less axial rotation can be accommodated. A rotational displacement about the longitudinal axis 60, in the range of between about 0° to about ±720°, more preferably to about ±360°, and even more preferably to about ±180°.

Generally, the selected boot material should not significantly transmit torque from the stent deployment assembly 45 to the proximal tubular shaft 43 during twisting. That is, a material should be selected that will not introduce any resistive torque in the direction opposite to the rotation of the stent deployment assembly. Another important quality of the protective boot material is that it is fluid impervious. By way of example, this protective boot 82 may be formed of a biocompatible, fluid impervious material, such as those composing balloon catheters. Further, with a sufficient wall thickness of about 10-100 microns, the protective boot will resist inflation during inflation of the dilator device should they be in common fluid communication with one another. This thin walled boot enables free rotation of the stent delivery assembly.

Briefly, one technique to achieve the required rotational properties of the balloon is to slightly pressurize the balloon material (e.g., 1 atm), and then twist the boot in an oscillatory manner (e.g., about 0-1000 times, more preferably about 0-100 times, and even more preferably about 0-50 times). This creates a plurality of small wrinkles in the boot material that facilitate rotation. This procedure should be performed prior to the cutting of the “balloon” sleeves to the correct length.

In another specific configuration, as shown in FIGS. 11 and 12, the boot design may be provided by a bellows-type balloon that permit axial rotation. In other instances, the balloon may be folded.

Referring now to FIGS. 11 and 13-14, another specific embodiment clutch assembly 48 is shown containing a plurality of interlocking tube elements 86, 86′ that cooperate for rotational displacement about the longitudinal axis 60. FIG. 9 shows elements 86, 86′ that are placed on a stainless steel cable 87 (e.g., stranded wires), or a steel wire, a Nitinol wire, polymeric “wire”, polymeric rod or any kind of reinforced rods i.e. polymeric reinforced rods with Carbon, glass or boron etc. Opposed annular bushing 88, 89 couple the clutch assembly 48 to the corresponding tubular shaft 43 and the transition portion 46. Preferably, although not limited to, the bushings 88, 89 are stainless steel, and can be produced from a single tube structure 135. FIG. 14 shows a flat view of geometry of the bushings for illustration purposes. The preferred production method will be laser cutting but is not limited to this. Instead of laser cutting other manufacturing procedures like micro EDM, etching or any kind of micro-machining can be used.

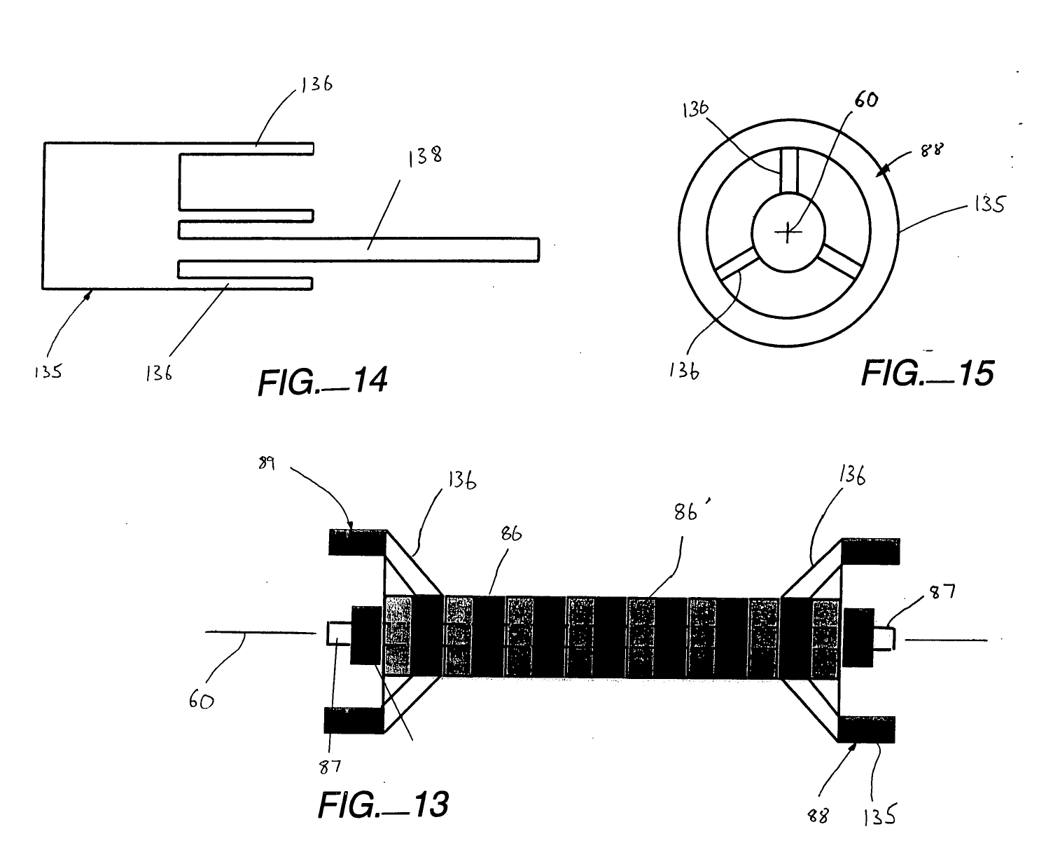

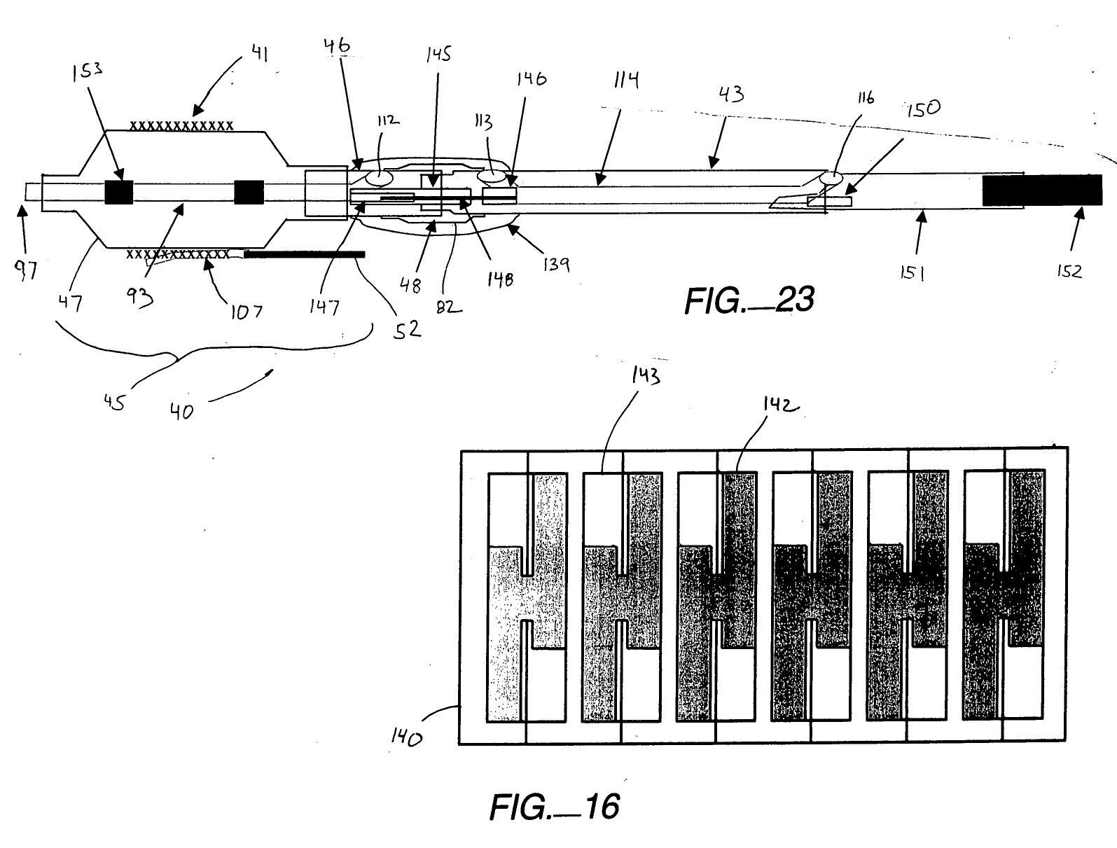

The flat structure 135 shown in FIG. 14, is actually annular-shaped (FIG. 15) with three arms 136 and optional an additional stiffening element 137. The three arms 136 are bent inwards and connected to one of the stainless steel elements 86, 86′ that are sitting on the steel cable 87 like pearls. Welding would be one sufficient connecting method but other methods like hot melt, soldering gluing etc. can be used as well. On each side proximally and distally one bushing will be connected. The outer diameter of the bushing will fit the inner diameter of the supporting tube 46 or the proximal tube 43. The bushings will be connected to the tubing 46 and 43 by any kind of connecting method like welding, shrinking gluing etc. To allow the bushings to fix in the tubing the distal end of the tube 43 and/or the proximal end of the supporting tube 46 can be enlarged to allow the bushing to fit in.

In order to provide a smooth transition on both ends or individually on the proximal or the distal end, a stiffening element 138 can be added at the entrance and exit point of the guide wire (FIG. 11). At these locations there will be no sufficient support since the guide wire is running on the outside of the catheter body.

This construction allows a maximum axial support of the catheter by having no limitation towards rotation. The resistance against rotation is minimal due to the PTFE or other low friction materials added to the chain construction. The clutch will be pressure sealed by a thin walled member that is running over the clutch and will only add minimal limitation towards rotation.

to decrease the resistant of the thin walled boot 82 over the clutch, the boot can be constructed like a bellow, as mentioned above. The additional folds in the boot will further minimize the resistance against torque.

The space between the arms of the bushings will be sufficient to allow fluid communication between the inflatable member and the proximal end of the catheter.

FIGS. 12 and 16 illustrate another specific embodiment having maximum axial support by minimal friction against rotation. FIG. 16 shows a flat view of a tubing 140 that is cut in a certain pattern. The cutting design allows the rings 141 to rotate against each other. The principle of this construction is a plurality of interlocking tube elements 142, 143 that cooperate for rotational displacement about the longitudinal axis 60. To prevent the elements 142, 143 from separating from one another, they will be placed over a PTFE or any other low friction tube. The interior of each element 142, 143 is to be a hollow construction to allow fluid communication between the inflatable member and the proximal end of the catheter.

Similar to the embodiment of FIG. 11, a pair of stiffening elements 144 can be added to the tube construction either by cutting indirectly from the tube or connecting into the tube element by laser welding or any other appropriate connecting method. As described above the stiffening element 144 will provide a smooth transition at the entrance and exit place of the guide-wire.

The proximal and distal end of the rotation tube will be connected to the supporting element 46 and the proximal tube 43. The distal end of tube 43 can be enlarged as well as the proximal end of the distal support tube 46 to fit the proximal and distal end of described rotation tube. In another embodiment the tubes 43 and 46 might fit directly to the size of the rotational tube or might even be tapered down.

Referring back to FIGS. 3 and 4, in order to rotationally align the substantially independently rotating stent deployment assembly 45 (about longitudinal axis 60) during advancement thererof along the guidewires 51, 52, the delivery assembly includes a set of guidewire passages (e.g., the first guidewire passage 90 and the second guidewire passage 91) that slideably receive the respective first and second guidewires 51, 52. At least a distal passage segment 92 of the first guidewire passage 90 extends centrally through the dilator device 47 generally along the longitudinal axis of the stent deployment assembly 45. The distal passage segment 92 of the first guidewire passage 90 is suitably sized for sliding receipt of a first guidewire 51 that is pre-advanced through the body vessel 42, typically through the main branch 53 as shown in FIGS. 3 and 4. In general, the distal passage segment 92 of the first guidewire passage 90 may be defined in part by a distal tube segment 93 of a first guidewire tube 95 extending at least through the expandable elements 96 (e.g., a balloon) of the dilator device 47. Hence, the first guidewire passage 95 and the distal passage segment 92 may be for the most part lumens. The distal passage segment 92 of the first guidewire passage 90 terminates at the distal end of the stent delivery device near its longitudinal axis thereof. A soft tip sleeve 97 may be mounted to the distal end of the distal tube segment 93 of the first guidewire tube 95 to as is commonly done in stent delivery systems in order to prevent vascular trauma during shaft advancement and to facilitate access through the lesion, and optimize tracking of the catheter.

In accordance with the present invention, the stent deployment assembly also includes a second guidewire passage 91 that extends in a direction generally adjacent to, although radially offset from, the first guidewire passage 90. As shown in FIGS. 3 and 4, the second guidewire passage 91 is also suitably sized for sliding receipt of a second guidewire 52 as the delivery assembly is advanced through the body vessel 42. Typically, a second guidewire 52 is employed through the body vessel 42 in situations requiring greater rotational alignment and deployment precision of the stent. In the examples illustrated herein, the second guidewire 52 is utilized to locate the side branch 55 of the bifurcated body vessel 42. As mentioned, a stent 41 loaded onto the delivery assembly must be accurately aligned with the side branch 55 for proper deployment.

The second guidewire passage 91 may be formed and/or fabricated along the stent deployment assembly using many different techniques as will be described below. One common physical characteristic, however, is that the second guidewire passage 91 extend under the collapsed stent 41 that is mounted over the dilator device 47 (FIG. 1A), and that a distal end or guidewire exit 98 from the second guidewire passage 91 be strategically positioned near or at the stent side branch port 103 of the collapsed stent 41. Briefly, the stent side branch port 103 will form the access port to the vessel side branch 55 when the stent is expanded and deployed. For example, the guidewire exit 98 from the second guidewire passage 91 terminates at the fish-mouth of the stent 41, radially offset from the first guidewire passage extending along the longitudinal axis.

In the particular embodiments illustrated in FIGS. 1A, 1B, 3 and 4, the second guidewire passage 91 is defined by a plurality of channels 100 formed between the exterior of non-inflated dilator device 47 and an underside of the particular struts 101 of the stent 41, in the collapsed state. Briefly, it will be appreciated that while the stent 41 is shown and illustrated more or less as a solid tube, the stent is actually composed of conventional patterned cells and expandable struts 101 similar to any other conventional stent.

Referring back to the configuration of FIGS. 1A and 1B, the second guidewire passage 91 extends longitudinally along an exterior of the non-inflated dilator device 47 in a direction substantially parallel to the first guidewire passage 90. More particularly, as shown in FIG. 1B, the corresponding struts 101 defining the corresponding channel 100 each include a smaller radius bend 102. It is notable, however, that an actually radius bend may not exist after the crimping process. Together with the non-inflated dilator device, the aligned channels 100 collectively define the second guidewire passage 91.

In accordance with the present invention, the position and off-set nature of the guidewire exit 98 of the second guidewire passage 91 relative to the first guidewire passage, as well as the independent, substantially resistant-free rotation of the stent deployment assembly collectively cooperate to self-align the dilator device 47 (and the strategically mounted stent thereon) with the vessel side branch 55.

Once the stent deployment assembly 45 nears the target site (e.g., from FIG. 3 to FIG. 4), where the first guidewire 51 and the second guidewire 52 diverge and are more distinctively separated (e.g., where tip of the first guidewire 51 extends into main branch 53 while the tip of the second guidewire 52 extends into the side branch 55), the off-set of the second guidewire exit 98 from the first guidewire passage 90 together with the substantially resistance-free rotation of the stent deployment assembly 45, via rotational clutch assembly 48, facilitate rotational alignment of the second guidewire passage 91 (about axis 60) toward the vessel side branch 55 (FIG. 4). In essence, as the stent deployment assembly 45 advances and tracks along the guidewires near carina 56, the second guidewire 52 diverges away from the first guidewire that extends down the vessel main branch 53. At the side branch 55 (directly above as shown in FIG. 4), the divergence of the second guidewire 52 away from the first guidewire 51 pulls the stent deployment assembly 45 into rotational alignment with the vessel side branch 55. Since the clutch assembly 48 provides substantially resistance-free rotation of the stent deployment assembly 45 about the longitudinal axis 60, it is more easily and precisely aligned with the side branch 55 for delivery and deployment of the stent.

When the stent deployment assembly 45 is advanced to the divergence between the first guidewire 51 and the second guidewire 52, further advancement of the system through the body vessel 42 will generally cease. At this position, the guidewire exit 98 of the second guidewire passage 91 will be rotationally aligned (about axis 60) with the vessel side branch 55 (aligned below in FIG. 4).

It will be understood, however, that the guidewire exit 98 of the second guidewire passage 91 could be located nearly anywhere longitudinally along the stent 41. Accordingly, depending upon the desired length of the extension of the stent 41 past the carina 56 and into the main branch 53 (which in-part may be dictated by the occurrence and position of any post branch lesion), the position of the stent side branch port 103, and thus, the guidewire exit 98 can be located nearly anywhere along the stent during manufacture or with a dedicated device during use. For example, if a longer extension of the stent 41, past the carina 56, is desired, the guidewire exit 98 can be positioned at a proximal portion of the stent 41. In contrast, by positioning the guidewire exit 98 of the second guidewire passage 91 closer to the distal end of the stent 41, a shorter stent extension into the main branch 53 is provided.

Once aligned, the dilator device 47 can be selectively inflated for radial expansion from a non-expanded condition (FIGS. 1, 3 and 5) to an expanded condition (FIGS. 5-8). Subsequently, the stent is expanded from a collapsed state to an expanded state for deployment in the bifurcation vessel. After expansion, the operator can decide how to proceed. In general, the guidewire 52 will be used to advance a second PTCA catheter into the region of the bifurcation.

The second balloon catheter has to pass through the struts of the stent. Once in place, the second balloon catheter can be inflated to dilate the untreated vessel. One advantage of this arrangement is that the delivery system of the first stent can remain in place even when an additional treatment is required.

After dilating the non-stented branch, the physician can further decide whether they want to place an additional stent in the other branch. The procedure will be normally finished by using the kissing balloon technique.

Briefly, while most types of bifurcation stents can be deployed in this manner, the delivery assembly of the present invention is particularly suitable for Provisional T type or fish-mouth stents, as above indicated. In this manner, the fish-mouth of the bifurcation stent 41 can be accurately aligned with the side branch 55 so that the side branch is not, or is minimally, occluded by the stent in any manner.

Furthermore, it will be appreciated that the delivery assembly of the present invention may be applied in combination with other devices and techniques that improve the precision and alignment of the delivery system with a bifurcated vessel. One such complementary system is that described in U.S. application Ser. No. ______, naming Von Oepen et al as inventors, filed May 4, 2006, entitled “GUIDEWIRE APPARATUS WITH AN EXPANDABLE PORTION AND METHODS OF USE”, and herein incorporated by reference in its entirety.

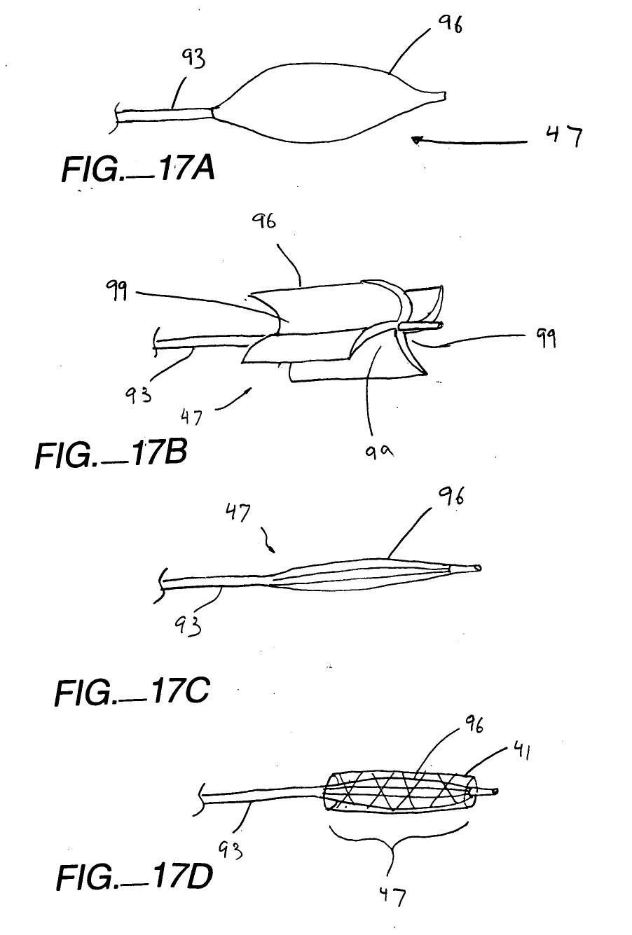

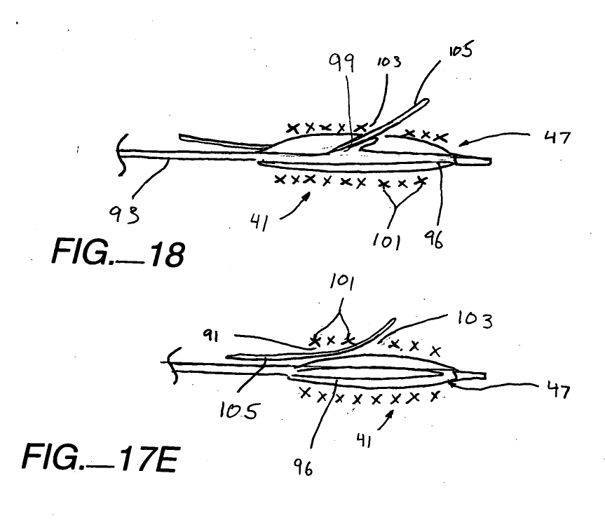

Turning now to FIGS. 17A-17E, the formation of the second guidewire passage 91, in the embodiments of FIGS. 1-4, is illustrated. Initially, as shown in FIG. 17A, the expandable elements 96 of the dilator device 47 are not folded. The expandable elements 96 are then folded using a conventional folding technique (i.e., as shown in FIG. 17C in the non-inflated condition). The folded, non-inflated dilator device 47 is then inserted into a partially expanded stent 41 (FIG. 17D).

For the specific embodiments of FIGS. 1-4, where the second guidewire passage 91 is formed between the underside of the stent 41 and the exterior of the expandable elements 96 of the dilator device 47, a mandrel 105 is inserted therebetween, as illustrated in FIG. 1 7E. This mandrel duplicates the desired path of the second guidewire passage 91. A proximal portion of the mandrel 105 enters the proximal end of the stent 41, while a distal portion of the mandrel exits through the stent side branch port 103 (or fish-mouth) of the stent 41. The stent in the as-cut condition 41 is then crimped onto the dilator device 47, using conventional crimping techniques, which forms the smaller radius bends 102 as the stent struts 101 and the expandable elements 96 are pushed against the mandrel.

Upon removal of the mandrel 105, which incidentally may be performed just prior to use, the second guidewire passage 91 is fabricated as shown in FIGS. 1A and 1B. In another configuration, the mandrel might be removed and exchanged against a transport stylet. It will be appreciated that upon expansion of the dilator device and deployment of the stent 41, the small radius bends 102 will be removed from the expanded structure of the stent.

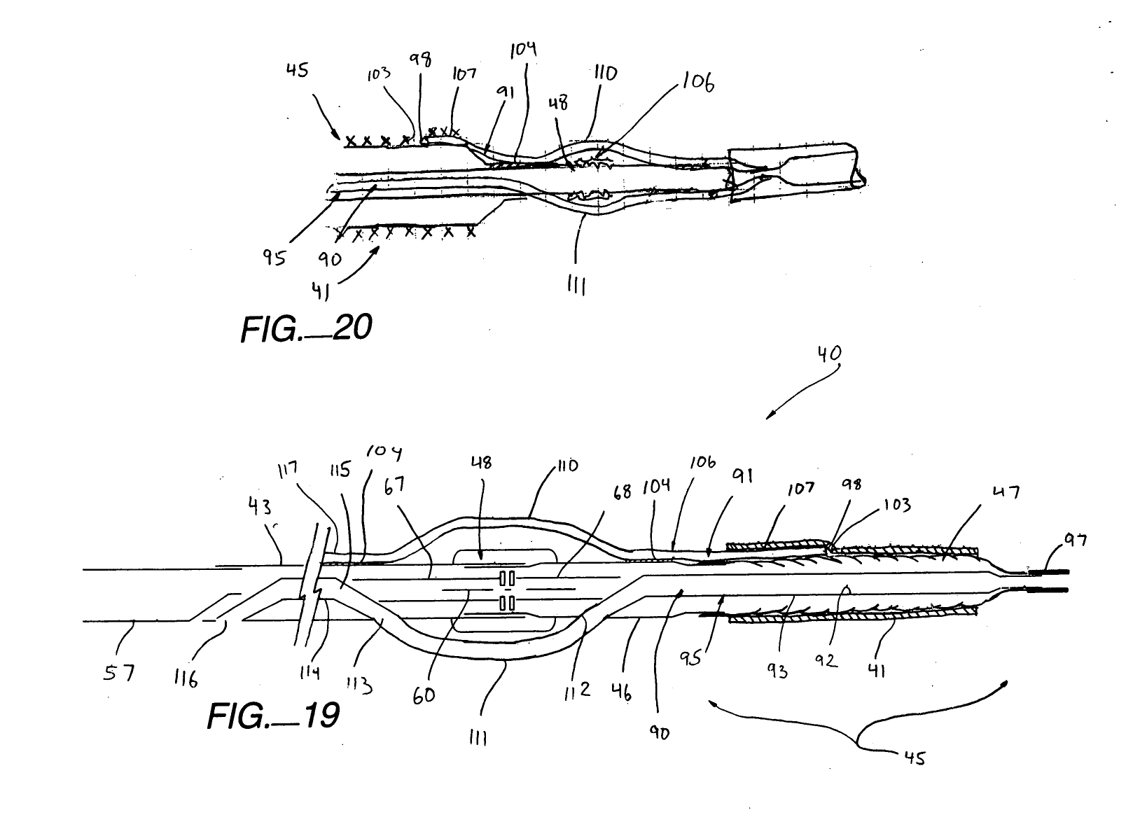

Alternatively, as illustrated in FIGS. 17B and 18, the mandrel 105 may be placed within a fold 99 of the expandable elements 96, under the exterior surface of the dilator device. Consequently, when the mandrel 105 is removed after conventional stent crimping, a second guidewire passage 91 will be formed that extends generally through the fold and under the exterior surface of the non-inflated dilator device.

In another specific embodiment, as shown in FIG. 19, the second guidewire passage 91 may be defined by a dedicated second guidewire tube 106 with a dedicated lumen. In this arrangement, a distal tube segment 107 of the second guidewire tube 106 extends through the proximal end of the stent 41 and between the exterior surfaces of the non-inflated dilator device 47. To even advance the alignment of the delivery system, it might be beneficial to provide a second catheter tip that is advanced through the stent struts. When this additional catheter tip is advanced into the side branch, it will facilitate the system to rotate in place. A distal end of the second guidewire tube, containing the guidewire exit 98, is preferably positioned proximate to the stent side branch port 103. Further, guidewire exit 98 of the distal tube segment 107 (also second guidewire passage 91) may actually be coincident, inside, or outside of the stent side branch port 103.

Similar to the formation of the second guidewire passage in the embodiments of FIGS. 1-4, the stent 41 may be crimped around the distal tube segment 107 of the second guidewire tube 106. Also similarly, the distal tube segment 107 may be interwoven or folded into the inflation elements of the dilator device itself for securement.

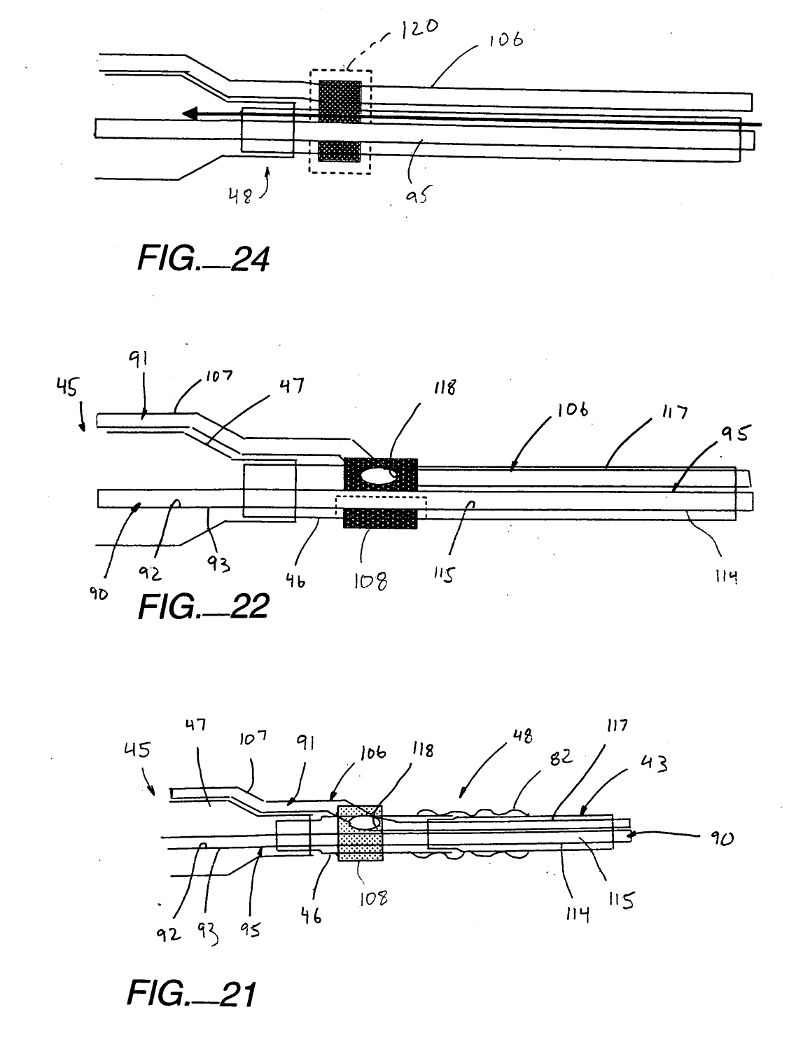

As viewed in FIGS. 19 and 20, the portion of the distal tube segment 107 of the second guidewire tube 106 extending adjacent the exterior of the distal transition portion 46 is also secured thereto using conventional techniques, such as an adhesive 104 or welding technique. In another embodiment shown in FIGS. 21 and 22, a support sleeve or band 108 may be applied affixing the distal tube segment 107 to the transition portion 46. Such sleeves or bands may exhibit elastic characteristics that resiliently couple a proximal portion of the distal tube segment 107 to the fluid-tight port 118.

It will be appreciated that distal tube segment 107 of the second guidewire tube 106 may be composed of a material that is capable of maintaining the integrity of the second guidewire passage 91 when being crimped against the dilator device 47, such as a crimping mandrel. The material of this tube segment, however, must also be sufficiently flexible to enable expansion of the dilator device 47 from the non-expanded condition (FIG. 19) to the expanded condition (FIGS. 20-24). Such materials include HDPE Polymide, PTFE, FEP, Polymide impregnated with PTFE, POM and other low friction plastics.

During operable use, when the first and second guidewires 51, 52 are being advanced through the corresponding first and second guidewire passages 90, 91 at the stent delivery assembly, the guidewires must both span or bridge across the rotatable clutch assembly 48 without interfering with the resistance-free rotational movement thereof. In the embodiments of FIGS. 1-4, the first guidewire 51 emerges from the distal tube segment 93 through a first port 112 in the transition portion 46 of the stent delivery assembly 46. Similarly, the second guidewire 52 emerges from a proximal end of a distal tube segment of the second guidewire passage 91 thereof. As best shown in FIGS. 3 and 4, both the first guidewire 51 and the second guidewire 52 extend loosely across the clutch assembly 48.

In the embodiment of FIG. 3, the second guidewire 52 extends further back and alongside the tubular shaft 43 once it passes through the passageway 91 of the stent delivery assembly. In another configuration (not shown), both the first guidewire 51 and the second guidewire 52 may extends alongside the tubular shaft 43 and all the way back once it passes through of the stent delivery assembly 45.

Alternatively, after bridging the clutch assembly 48, none, one or both loose guidewires 51, 52 may subtend into a proximal passage segment 115, 115′ of their respective guidewire passages 90, 91, and through corresponding second and third ports 113, 113′ extending into the tubular shaft 43 (FIG. 4). As shown in FIG. 3 (as well as the embodiments of FIGS. 5-7), for example, only one guidewire (e.g., the first guidewire 51) subtends into the tubular shaft 43, via the second port 113. In another specific arrangement, as shown in FIG. 4, both the first and second guidewires 51, 52 subtend into the tubular shaft 43, via the second and third ports 113, 113′, respectively.

Both ports 113, 113′ are preferably situated at a location proximal to the clutch assembly 48. Further, to aid insertion and passage of the loose guidewire 51, 52 into the respective port 113, 113′, a guidewire loading tool may be used that incorporates a hood or shield positioned over or proximate to the port.

In the configuration of FIGS. 3 and 4, a proximal tube segment 114 of the first guidewire tube 95 is disposed longitudinally in the tubular shaft 43. The proximal tube segment 114 defines a proximal passage segment 115 of the first guidewire passage 90 that is coupled to the second port 113. This proximal tube segment 114 is coupled to fourth access port 116 that provides access to the lumen through the adapter 57. Similarly, in the embodiment of FIG. 4, the second guidewire tube 106 includes a proximal tube segment 117 that is disposed longitudinally in the tubular shaft 43. The proximal tube segment 117 defines a proximal passage segment 115′ of the second guidewire passage 91 that is coupled to the third port 113′.

In one specific example, as shown in FIG. 19 and 20, both the first guidewire passage 90 and the second guidewire passage 91 are defined in-part by exterior loose tube segments 110, 111 that span the clutch assembly 48 from the tubular shaft tubular shaft 43 to the transition portion 46, and permit relative rotation therebetween. Such tubular span sections facilitate advancement of the respective guidewires into the distal segments of the respective guidewire passages 90, 91, as well as enclose the passages across the span of the clutch assembly. In the embodiment of FIG. 19 the second guidewire tube 106, defining the second guidewire passage 91, bridges the gap and then extends all the way along and adjacent to the tubular shaft 43. As mentioned, a distal segment 107 of the second guidewire tube 106 is mounted to the exterior surface of the transition portion 46, just distal to the clutch assembly for stability (e.g., by adhesive 104). Similarly, the proximal tube segment 117 of the second guidewire tube 106 is mounted to the exterior surface of the tubular shaft 43, just proximal to the clutch assembly also to enhance stability (e.g., by adhesive 104). In fact, the entire proximal tube segment 117 of second guidewire tube 106 may be adhered to or melded with the tubular shaft 43 for security thereof during advancement through the body vessel.

In accordance with the present invention, however, a loose tube segment 110 of the second guidewire tube 106, bridging or spanning the clutch assembly 48 must have a sufficient length, and/or include the ability to permit substantially resistance-free rotational displacement of the stent deployment assembly 45 about the clutch assembly longitudinal axis 60.

In a similar manner, as shown in FIG. 19 (as well as the embodiments of FIGS. 5-7), the first guidewire tube 95 also includes a loose tube segment 111 spanning the clutch assembly 48 that is also of sufficient length and characteristics to enable substantially resistance-free, interference-free rotation of the stent deployment assembly 45, about axis 60, relative to the tubular shaft 43. In contrast to the second guidewire tube 106, the distal tube segment 93 of the first guidewire tube 95 extends into an interior portion of the transition portion 46 and the dilator device 47 of the deployment assembly 45.

This loose tube segment 111 of the first guidewire tube emerges from the first port 112 to extend exteriorly across the clutch assembly 48. In the configuration of FIG. 19, the first guidewire tube 95 enters the tubular shaft 43 at a location proximal to the clutch assembly 48, through a fluid-tight second port 113. A proximal tube segment 114 of the first guidewire tube 95 extends through the tubular shaft 43. This proximal tube segment 114 defines a proximal passage segment 115 of the first guidewire passage 90 that provides access thereto through a third port 116.

It will be appreciated that while the first guidewire tube 95 and the second guidewire tube 106 have each been described as being essentially one continuous tube, they may be defined by multiple components that collectively form the respective tubes and their corresponding lumens. For instance, in the embodiment of FIG. 19, the first guidewire tube 95 may terminate at the second port 113, and the proximal passage segment 115 of the first guidewire passage 90 may be defined by internal structure of the tubular shaft 43 itself.

In another specific embodiment, as shown in FIG. 20, respective proximal tube segments 114, 117 of the first and second guidewire tubes 95, 106, respectively, extend entirely along the exterior of the tubular shaft 43. As mentioned, these proximal tube segments 114, 117 may be mounted or adhered to the tubular shaft for stability during vessel advancement (e.g., adhesive 104).

In another embodiment not shown, the proximal tube segment 117 of the second guidewire passage 91 may subtend into the tubular shaft 43, and extend internally therethrough. Similar to those embodiments for the first guidewire tube 95, another fluid-tight port may be provided just proximate to the clutch assembly 48 than enables passage into the tubular shaft 43.

FIGS. 21 and 22 illustrate yet another embodiment showing the first guidewire passage 90 contained entirely within the stent delivery system. In this configuration, for example, the first guidewire tube 95 may extend through the tubular shaft 43, the clutch assembly 48, and through the stent deployment assembly 45. In contrast, in this embodiment, the distal tube segment 107 of the second guidewire tube 106 exits the transition portion 46, distal to the clutch assembly 48, through a fluid-tight fourth port 118. As previously indicated, the distal tube segment 107 of the second guidewire tube 106 then extends along a portion of the exterior of the transition portion and along at least a portion of the dilator device 47. As shown, the second guidewire passage 91 also extends through the clutch assembly 48, and through the tubular shaft.

In another embodiment, as shown in FIG. 23, an outer flexible cover member or outer protective boot 139 may further be disposed about the inner protective boot 82. This cylindrical-shaped boot 139 is sufficiently long to span and enclose both the first port 112 and the third port 113. Hence, this section of the first guidewire passage 90 is essentially enclosed by the outer protective boot 139 similar to loose tube segment 111 of the embodiments of FIGS. 5-7. The proximal and distal portions of the outer boot 139 may be affixed to the outer surface of the tubular shaft 43 and the transition portion 46, respectively, in a manner similar to that of the inner boot 82. Further, the flexibility characteristics and properties should be similar as well to enable relatively resistance-free rotation of the deployment assembly 45 relative to the tubular shaft 43.

This configuration also illustrates an interior first reinforcement tube 145 spanning the clutch assembly 145 generally from the first port 112 to the third port 113. An interior second reinforcement tube 146 is disposed proximate to the third port 113 that is spaced-apart from and smaller in length than the first reinforcement tube 145. The first reinforcement tube 145 includes an interior pocket 147 formed to receive a centrally disposed stiffening wire 148 that spans the gap from the first tube 145 to the second tube 146 where it is also interiorly received. Similar to the embodiment of FIG. 8, this configuration promotes axial stiffness while permitting relative rotation of the clutch assembly 48 about the longitudinal axis.

A third reinforcement tube 150 is disposed at the intersection or joint between the tubular shaft 43 and the middle tube 151. This joint defines the fourth access port 116 of the proximal tube segment 114. This reinforcement tube also promotes axial stiffness during advancement of the device. Typical materials of all the reinforcement tubes include Nitinol, stainless steel, PEEK, and carbon fiber, for example. A hypo tube 152 may be mounted to the proximal end of the middle tube 151. Furthermore, spaced-apart RO markers 153 are disposed about at the distal tube segment 93 of the first guidewire tube 95, which facilitate positioning of the stent delivery assembly 45.

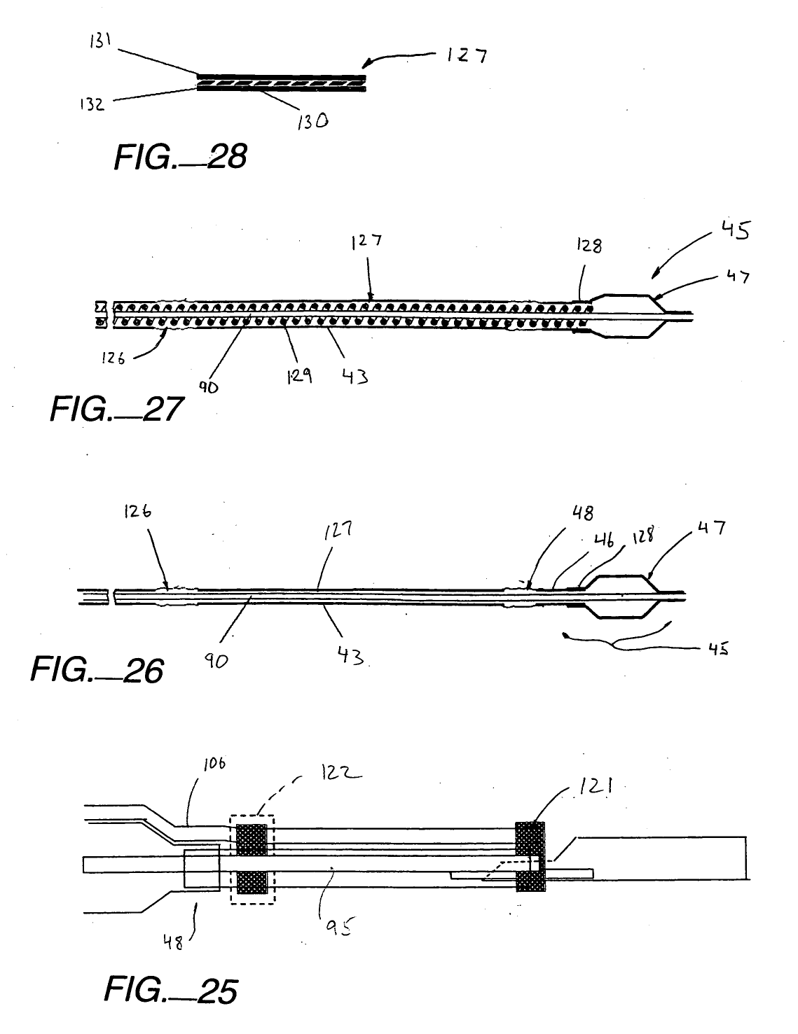

Referring to FIGS. 24 and 25, a double arm catheter 120 is illustrated in which the second guidewire tube 106 is independent in construction from the first guidewire tube 95. In FIG. 24, the second guidewire tube 106 and first guidewire tube 95 are connected in a location proximal to the clutch assembly 48, for example, by a band 120. This allows the guidewire tubes to bend independently of each other.

In FIG. 25, a configuration is shown in which the independent guidewire tubes 95, 106 are connected in two locations proximal to the clutch assembly 46, for example, by a proximal band 121 and a distal band 122. In this configuration, the guidewire tubes can bend independently within the space between the connections.

In yet another specific embodiment, as exemplified in FIGS. 26 and 27, a second rotational clutch assembly 126 may be included that provides additional rotational dampening, similar to the first clutch assembly 48. This second clutch assembly 126 is positioned proximal to the first clutch assembly 48.

In still another specific embodiment, a torque transmitting device 127 may extend through the entire length of the tubular shaft 43 and through clutch assembly 48 to the stent deployment assembly 45. As shown in FIGS. 27 and 28, this torque transmitting device 127 includes a distal portion mounted to a proximal shoulder 128 of the dilator device 47. This torque transmitting arrangement functions to transmits torque to the stent deployment assembly 45 for limited control and orientation thereof.

In one embodiment, the torque transmitting device 127 may be provided by a braided inner shaft. In another configuration, as shown in FIG. 27, the torque transmitting device 127 may includes a spiral wire 129 with any cross-sectional shape, such as a round, oval, or rectangular cross sectional area extending through the stent delivery system 40. The spiral wire 129 will be coupled to a hypotube or stiffening wire of the proximal portion of the tubular shaft 43. The distal end of the spiral wire, as mentioned, will be mounted to the proximal shoulder 128 of the dilator device 47.

In another configuration of FIGS. 26 and 28, the torque transmitting device 127 may be provided by a flat wire 130 that reinforces the inner lumen of the inner tube. A nylon liner 131 and a PE liner 132 cooperate with the flat spring wire reinforcement to transmit torque in on preferred direction.

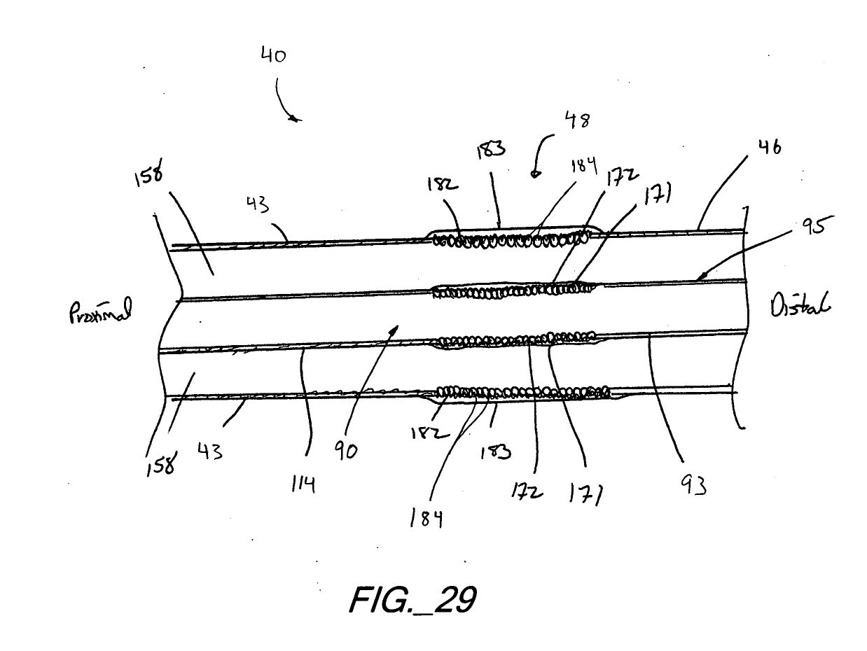

Referring now to FIG. 29, there is shown yet another specific embodiment of the clutch assembly in accordance with the present invention. In this specific arrangement, the clutch assembly 48 includes an outer flexible member 182 coupling the distal portion of the tubular catheter shaft 43 to the proximal portion of the tubular transition portion 46, as well as including an inner flexible member 172 coupling the distal portion of the proximal tube segment 114 to a proximal portion of the distal tube segment 93, the latter of which cooperate to define a portion of the first guidewire passage 90 of the delivery system 40.

In accordance with this specific embodiment of the present inventive clutch assembly 48 of FIG. 29, one or both of the outer flexible member 182 and the inner flexible member 172 may be provided by a wound structure, disposed relatively co-axial to one another. Each wound structure must be capable of permitting relatively interference-free rotational displacement between the outer tubular catheter shaft 43 and the outer tubular transition portion 46, about longitudinal axis 60, as well as between the inner proximal tube segment 114 and the distal tube segment 93. More particularly, one end of the outer flexible member 182 is fixedly mounted to the end of outer tubular catheter shaft 43 while the other opposite end is fixedly mounted to the outer tubular transition portion 46. Similarly, one end of the inner flexible member 182 is fixedly mounted to a distal end of inner proximal tube segment 114 while the other opposite end is fixedly mounted to the distal tube segment 93. For a wound structure, for example, the end coils would thus be mounted to their respective tubular components.

The outer flexible member 182 may be constructed of a single wound coil or multiple wound coil shaped spring in a nested configuration that is composed of a metallic material such as stainless steel, nitinol, platinum, gold, silver or similar materials. Alternatively, the wound member may be constructed of non-metallic materials such as nylon, PVC, Pebax or similar bio-compatible materials. The wound member 182 may also be constructed by winding a flexible material about a mandrel as is well known in the art.

Accordingly, such a wound type structure not only permits relatively interference-free rotational axial displacement about the longitudinal axis 60, but also promotes axial stiffness. The adjacent coils 184, hence, must be closely spaced if not in contact with one another when a compressive axial force is applied thereto during advancement.

As best shown in FIG. 29, a cylindrical-shaped outer sealing member 183 may be disposed about the outer flexible member 182 to provide a fluid tight seal between the outer surface of the tubular shaft 43 of the delivery system 40 and the inner lumen 158. Similar to the previously described embodiments, the inner lumen 158 is utilized as an inflation lumen for an expandable member, such as a balloon, disposed adjacent the clutch 48. This outer protective boot or outer sealing member 183 may be constructed of a material such as silicone tubing, nylon, urethane, pebax, or similar materials that are biocompatible and capable of being affixed to the outer surface of the proximal portion and distal portion of the catheter. The outer sealing member 183 may be a tubular member that has been disposed over the outer flexible member 182 or alternatively, the outer flexible member 182 may be dip coated or spray coated with a selected material to form a fluid tight coating. In yet another embodiment not shown, an inner sealing member may be disposed about the outer flexible member 182, wherein one end of the inner sealing member would be affixed to the inner wall of the tubular shaft 43 while an opposite end thereof would be affixed to the inner wall of the tubular transition portion 46 to provide a fluid tight seal between the outer flexible member 182 and the lumen 158 of the delivery system 40.

As mentioned, this specific embodiment of the clutch assembly 48 further includes an inner flexible member 171 disposed between the proximal tube segment 114 and the distal tube segment 93. These co-axially aligned tube segments 114, 93, together with the inner flexible member 171 defining a distal portion of the first guidewire passage or lumen 90 of the delivery system 40. The inner flexible member 171 may be constructed of a material such as those described previously with regard to the outer flexible member 182. As described above, the inner flexible member 171 may be coated with a material such as those described above in order to maintain a fluid tight lumen disposed between the inner flexible member and the outer flexible member. Hence, the annular lumen 158 therebetween can be configured as an inflation/deflation lumen for an expandable member disposed on a distal portion of the catheter. An alternative to coating the inner flexible member is to provide a sleeve of material about the flexible member and affixing the ends of the sleeve to the tubular member disposed on either side of the inner flexible member. Suitable materials of which the sleeves may be formed include silicone, PVC, nylon, urethane, pebax and blends thereof.

As shown in FIG. 29 and as mentioned, the opposed ends of the inner flexible member 171 are coupled to the respective ends of the inner proximal tubular segment 114 and the inner distal tubular segments 93. Any conventional mounting techniques can be applied that enable the tubular segments and the flexible member to function as a guidewire lumen. For example, the ends of the inner tubular segments adjacent the location of the inner flexible member 171 ends may be flared, necked down or otherwise reduced/enlarged in diameter. Alternatively, a complementary spiral pattern may be formed in the thickness of the ends of proximal/distal tubular segments 114/93 or through the wall of the tubular segments. This configuration would allow the inner flexible member 171 to be threaded into the ends of the inner tubular segments 114/93, wherein the coating/sleeve 172 may be applied to affix and secure the inner flexible member 171 to the ends of the respective tubular segments.

It is further contemplated that the inner flexible member 171 and the corresponding tubular segments may be constructed from a unitary member. That is, the spiral formation of the flexible section may be formed using known manufacturing processes such as cutting, laser cutting, water jet cutting and other similar processes.

The sleeve/coating 172 itself may also be fixedly attached to the ends of the respective inner proximal/distal tubular segment 114/93 through the use of known attachment methods. For example, the sleeve /coating may be melted to the outer surface of the inner tubular segments, or fastened through the application of adhesives and/or mechanical fasteners such as crimping a band of metallic material.

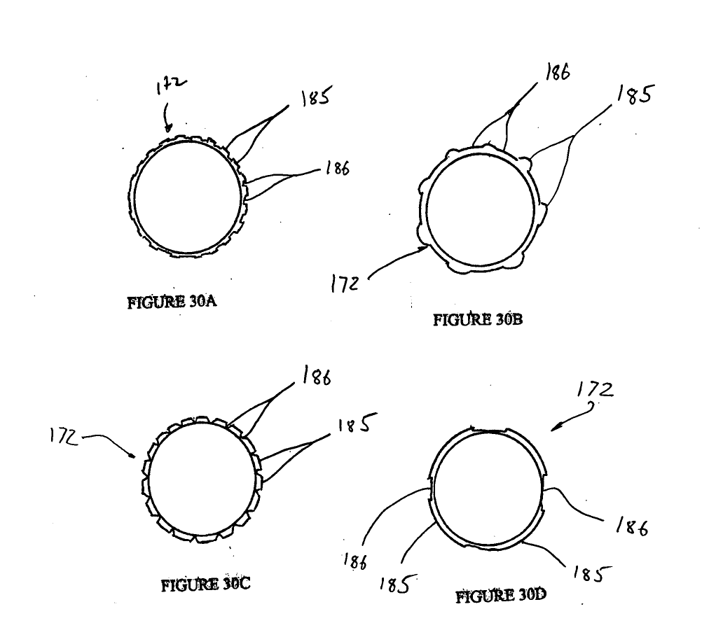

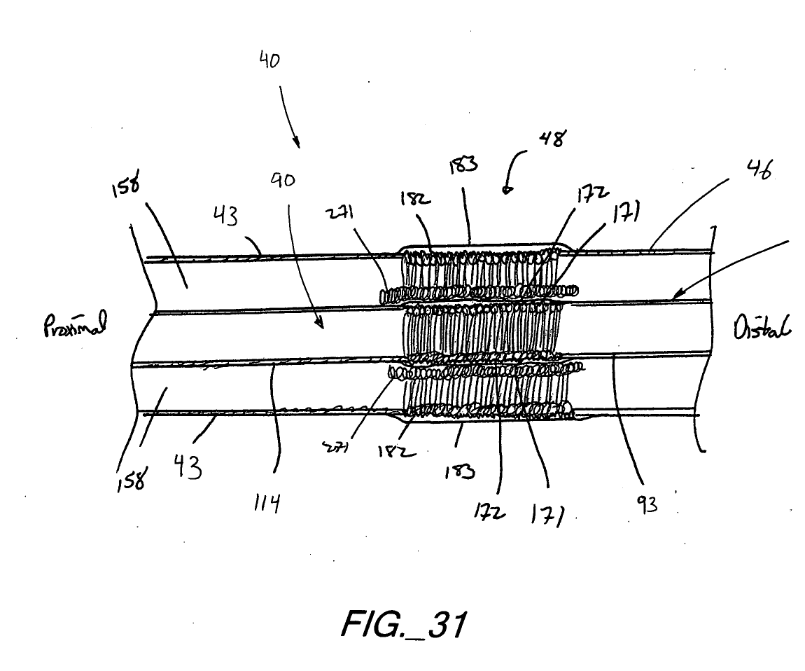

To substantially reduce or prevent collapse of the outer flexible member 182 onto the inner flexible member 171 under a vacuum, such as during fluid preparation of the device or deflation of the expandable member, the coating and/or the sleeve 172 applied to the inner flexible member 171 may further include a stand-off feature 185 formed therein. Referring now to FIGS. 30A through 30D, there are shown exemplary cross-sectional views of alternative embodiments of features 185 that may be formed within or upon the outer wall of the coating and/or sleeve member 172. Generally, such stand-off features form a protrusion extending radially outward from an outer peripheral surface of the coils 184 of the inner flexible member 172. Any pattern of such radially-spaced protrusions or features 185 are sufficient so as to form a communication channel 186 between the adjacent protrusions that extends longitudinally from one end of inner flexible member to an opposite end thereof. Under a vacuum, for instance, the outer flexible member 182 may collapse and come to rest upon or abut against the protrusions 185 while the communication channels 186 formed therebetween enable fluid communication across the clutch assembly 48, in the inflation /deflation lumen 158. Hence, the entire collapse of the outer flexible member 182 onto the inner flexible member 171 that might prevent fluid communication in the inflation/deflation lumen 158 is averted.

Another manner in which to address a collapse of the outer flexible member 182 onto the inner flexible member 171 under vacuum is through the disposal of an additional central flexible member 271, or as shown in FIG. 31, two central flexible members 271 in the inflation/deflation lumen 158 between the outer and inner flexible members 182 and 171. These central flexible members 271 are disposed longitudinally in the lumen 158, and have a diameter much small than that of the outer and inner flexible members. Each central flexible member 271 includes a first end, a second end and a lumen disposed therebetween. The inner flexible member(s) 271 may be constructed in the same manner as described with regard to the outer flexible member 182 and the inner flexible member 171 (i.e., as a wound member).

In use, under vacuum, the central flexible member(s) 271 prevent the outer flexible member 182 from touching or becoming stuck to the coating 172 applied to the outer surface of the inner flexible member 171. Similar to the features 185 above, during collapse of the outer flexible member 182, under vacuum, the inner, outer and central flexible member will cooperate to form a communication channel therebetween in the lumen 158 that provides sufficient fluid communication.

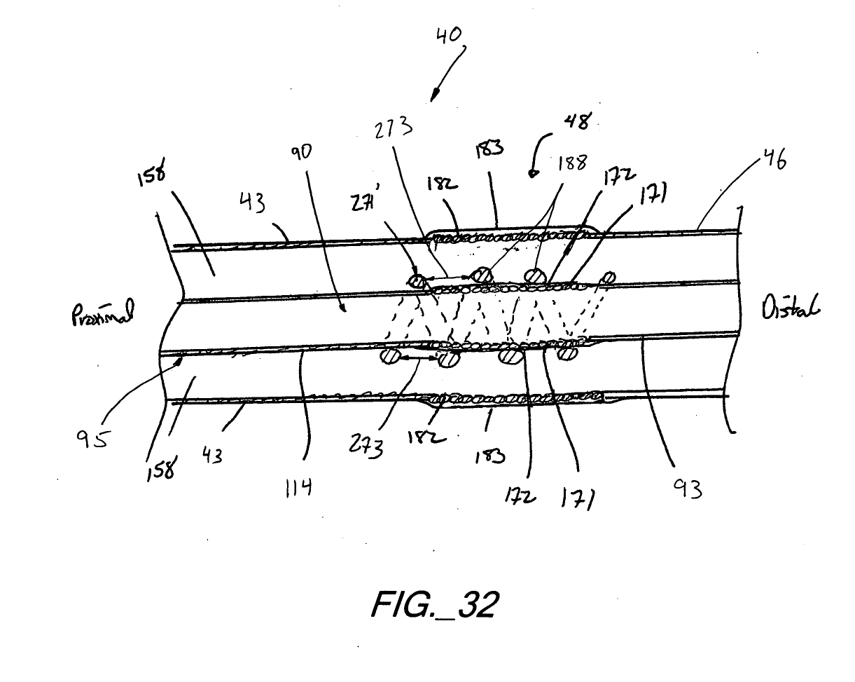

Referring now to FIG. 32 there is shown yet another embodiment of the central flexible member 271′, in accordance with the present invention. As described above, it may be desirable to place an additional flexible member within the fluid lumen 158 in order to ensure the fluid lumen 158 remains patent during use. In this particular embodiment, a central flexible member 271′ is formed that is disposed in the fluid lumen 158 about the inner flexible member 171. Hence, the central flexible member 271′ includes a central lumen sized for receipt of the inner flexible member 171 therein. Similar to the inner and outer flexible members described above, this embodiment of the central flexible member is also a wound member. In the embodiment shown in FIG. 32, the central flexible member is disposed within the lumen 158 having adjacent coils 188 sufficiently spaced-apart in a stretched manner such that there is a fluid channel 273 is formed between adjacent coils 188. These spiral-shaped fluid channels 273 provide a communication path for which fluid used for inflation of a balloon disposed distal the clutch 148 may flow. Additionally, under vacuum, the channels 273 ensure a patent path for the fluid to flow within the lumen 158.

As described above, the clutch assembly 48 of the present invention allows the distal section of the catheter in accordance with the present invention to rotate independent of the proximal portion of the catheter. Advantages of the independent inner and outer flexible members include the ability of the stent deployment assembly 45 of the delivery assembly 40 to rotate freely of the tubular shaft 43 thereof as previously described. Additionally, the design of the flexible members, while allowing independent rotation of the proximal and distal sections of the catheter allows an axial force translated longitudinally along the length of the catheter to be transmitted.

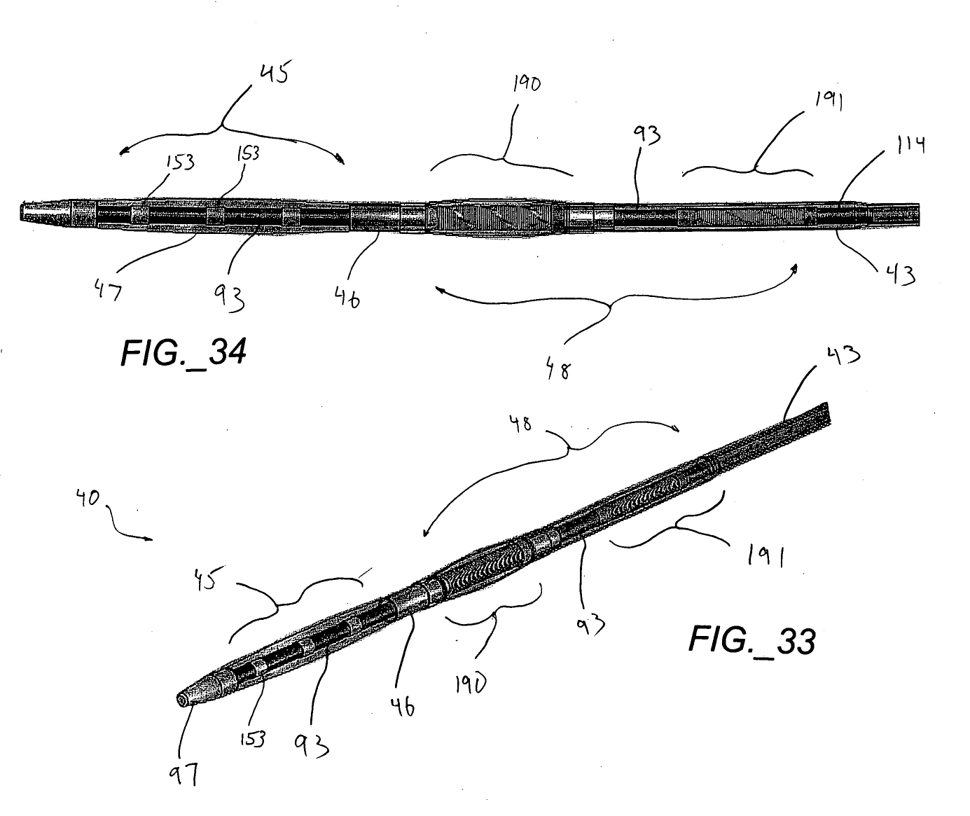

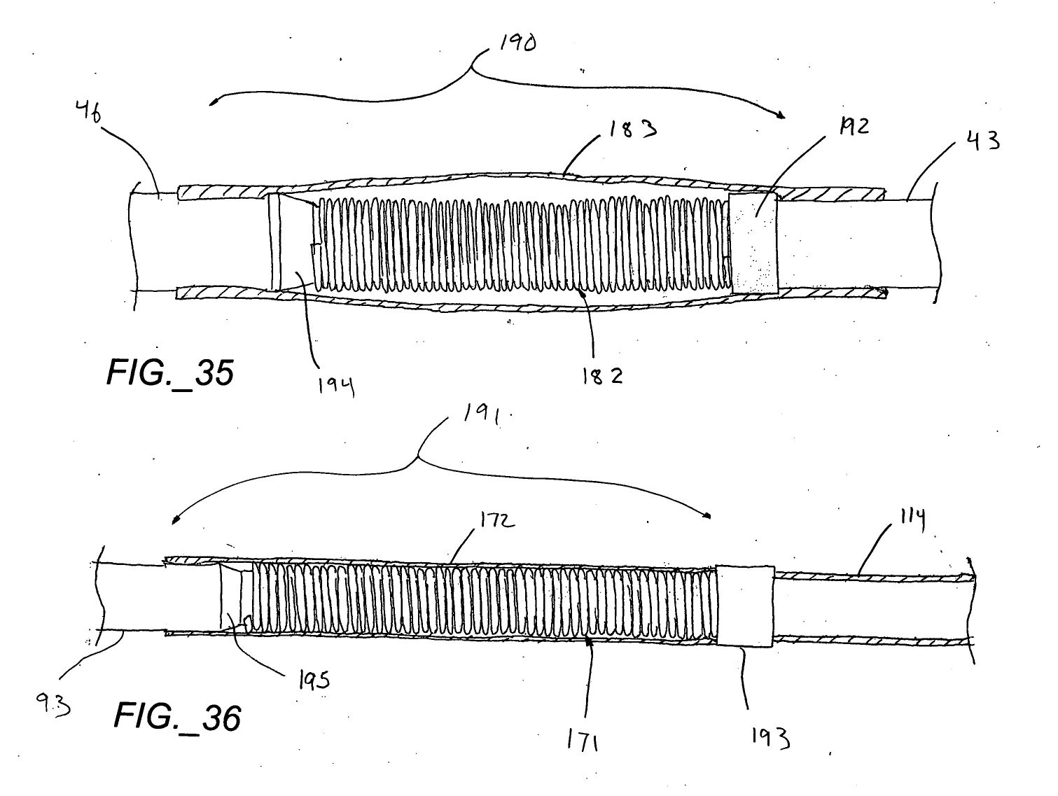

Turning now to FIGS. 33-36, another specific embodiment is illustrated incorporating an axially staggered arrangement of the inner flexible member 171 relative to the outer flexible member 182. Accordingly, the clutch assembly 48 essentially consists of an outer clutch device 190 and an inner clutch device 191. Typically, the outer clutch device 191, as we the previous embodiments, is the component incurring a substantially portion of the torsion loads and axial loads during operation. The two clutch devices can in fact operate relatively independent of one another. Such an axial offset is also beneficial in that the overall profile can be reduced since the inner and outer flexible members are not nested. Moreover, as will be described, this arrangement prevents buckling since, using either the elongated shaft 43 to support the inner flexible member 171, or the inner distal tube segment 93 to support the outer flexible member 182. Preferably, both flexible members should be positioned relatively close to the stent deployment assembly 45, this should not be limiting. Moreover, while it is preferable to place the outer flexible member 182 closer to the stent deployment assembly 45, it will be appreciated that the inner flexible member 171 may reside closer to the deployment assembly than the outer flexible member.

In this specific embodiment, the proximal portions of both the outer and inner flexible members 182, 171 are fixedly mounted to their respective proximal tube segment 114 and the elongated shaft 43, respectively, through a respective support ring 192, 193. Such rings provide additional axial support to the corresponding flexible members at their proximal ends as well as providing a means for mounting the coiled members to their respective tube segment and elongated shaft.

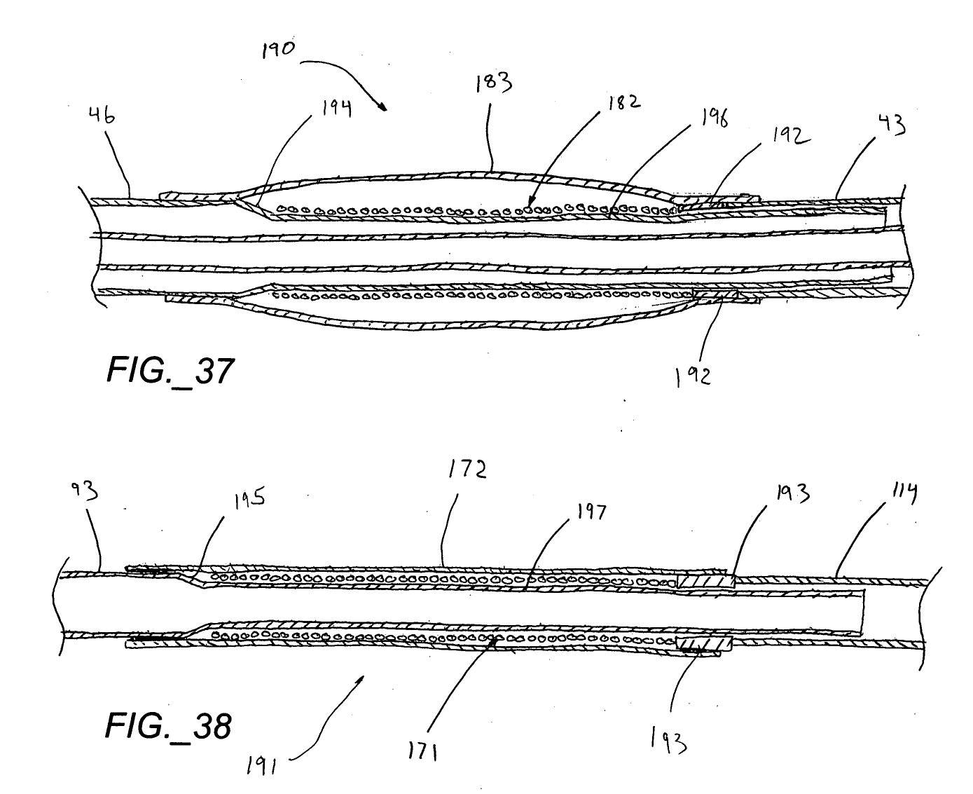

In contrast, in this configuration, an opposite distal end of the outer flexible member 182 and the inner flexible member 171 is not affixed to the respective tubular distal tube segment 93 and transition portion 46. As best viewed in FIGS. 37 and 38, each proximal portion of the transition portion 46 and the distal tube segment 93 includes an inward taper portion 194, 195 that is coupled to a respective inner support shaft 196, 197 sized to axially pass through the respective flexible member 182, 171 and terminates at a location proximal thereto. These inner support shafts not only provide additional lateral stability, but also provide support upon which each respective flexible member can rotate about.

Accordingly, both the outer clutch device 190 and the inner clutch device 191 permit limited axial displacement between the respective shafts or tube segments that they associate with. During advancement of the delivery system 40 through a body vessel, compressive axial displacement will be limited when the distal end of the respective outer flexible member 182 and the inner flexible member 171 abut and engage the respective taper portions 194, 195. Accordingly, the tapered portions 194, 195 must be sized and dimensioned to prevent slippage of the distal ends of the respective flexible members 182, 171 distally beyond the tapers.

In contrast, during retraction of the stent delivery system 40 from the body vessel, it is the corresponding protective sleeve or boot 188, 172 that substantially bears the tensile loads. Since the outer clutch device 190, as mentioned, is subject to more significant torsion and axial loads under operation, the outer protective boot 183 is preferably configured to be more durable than that of the inner protective boot 172. Accordingly, a more durable material, such as a Pebax or the like is selected to withstand the twisting and tensile loads it will endure during use. Moreover, the boot is more loosely fit about the corresponding outer flexible member 182 to enable more significant relative rotational displacement. In contrast, the inner protective boot 172 may be composed of a silicon material or the like that is thinner and more form fit around the inner flexible member 171.

It is further contemplated that an additional stiffening member may be incorporated in all these embodiments, such as the inner support shafts of the embodiments of FIGS. 33-38, longitudinally extending across either the inner or outer flexible member, or both, to enhance the transmission of longitudinal or axial forces. The additional member may be in the form of an additional flexible member as described above, or alternatively may be strictly a stiffening element constructed of a longitudinal member.

In accordance with the present invention, the flexible members embodied in the form of a wound member may be disposed in either a clockwise, counterclockwise orientation or in a combination of either of the two orientations. Further, the wound flexible members may be provided with a variety of pitches and torsion rates, although all must permit rotation about their longitudinal axis with very small rotational forces.

The invention is susceptible to various modifications and alternative forms, and specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the claims.

What is claimed is:

1. A stent delivery system for delivering and deploying a radially expandable stent at a strategic orientation and location in a body vessel, said delivery system comprising:
an elongated shaft;
a stent deployment assembly including a proximal transition portion associated with a dilator device adapted for radial expansion from a non-expanded condition to a radially expanded condition, said dilator device configured to retain said stent in the non-expanded condition; and
a rotational clutch assembly configured to rotatably mount the transition portion to a distal portion of the elongated shaft such that said deployment assembly is substantially torsionally isolated from said elongated shaft.

2. The stent delivery system according to claim 1, wherein
said clutch assembly is adapted to transmit compression forces longitudinally along the distal portion of the elongated shaft to the deployment assembly during advancement of the elongated shaft through the body vessel.

3. The stent delivery system according to claim 2, wherein
said clutch assembly includes an inwardly tapered shoulder portion coupled to one of a distal end of the elongated shaft and a proximal end of the transition portion, said clutch assembly further including a neck portion extending from said tapered shoulder portion, said neck portion being formed and dimensioned for sliding rotational receipt into an opening at the other of the tubular transition portion and the elongated shaft for rotational receipt thereof.

4. The stent delivery system according to claim 1, further including:
a flexible protective boot device extending circumferentially over the clutch assembly having one end secured to the elongated shaft and an opposite end secured to the transition portion such that a fluid-tight seal is formed while still enabling relative rotation between the elongated shaft and the deployment device.

5. The stent delivery system according to claim 1, further including:
said stent deployment assembly defining at least a portion of a first guidewire passage therethrough, said first guidewire passage being sized and dimensioned for sliding receipt of a first guidewire disposed in said body vessel.

6. The stent delivery system according to claim 5, further including:
a second guidewire passage extending along at least a portion of said stent deployment assembly, and terminating strategically along the dilator device of said stent deployment assembly, said second guidewire passage being sized and dimensioned for sliding receipt of a second guidewire disposed in said body vessel, and said second guidewire passage being off-set from said first guidewire passage such that during advancement along said first and second guidewires in the body vessel, said deployment assembly will be caused to rotate into alignment with the position of the second guidewire relative to the first guidewire.

7. The stent delivery system according to claim 6, further including:
a distal guidewire tube segment defining a distal segment of said second guidewire passage, said tube segment being secured to the transition portion for rotation thereof about the longitudinal axis of the deployment assembly, and having a distal end terminating along an exterior of said dilator device.

8. The stent delivery system according to claim 7, wherein
a distal portion of said distal guidewire tube segment being disposed between the stent and the dilator device in the non-expanded condition.

9. The stent delivery system according to claim 2, wherein said clutch assembly includes a pair of opposed contact elements disposed in opposed relationship to one another, one contact element being associated with the elongated shaft while the second contact element being associated with the transition portion such that during said advancement of the elongated shaft through the body vessel, the contact element are moved into compressive mutual contact with one another to transmit axial compressive forces from the elongated shaft to the transition portion.

10. The stent delivery system according to claim 9, wherein
said clutch assembly includes a first support tube associated with the elongated shaft, and a second support tube associated with the transition portion, each the first and second support tube having respective end portion substantially in opposed relationship to one another, each end portion supporting one of said contact elements in opposed relationship to one another.

11. The stent delivery system according to claim 10, wherein
said clutch assembly further includes an elongated stiffening element extending substantially longitudinally thereacross, one end of said stiffening element being disposed in a distal pocket defined in part by a distal end wall of the transition portion, and an opposite end of said stiffening element being disposed in a proximal pocket defined in part by a proximal end wall of the elongated shaft such that during said advancement of the elongated shaft through the body vessel, one end of the stiffening element contacts the distal end wall and the opposite end of the stiffening element contacts the proximal wall to transmit axial compressive forces from the elongated shaft to the transition portion.

12. The stent delivery system according to claim 11, wherein
said clutch assembly includes a first support tube associated with the elongated shaft and defining said distal pocket, and a second support tube associated with the transition portion and defining said proximal pocket, each the first and a second support tube having respective end portion substantially in opposed relationship to one another.

13. A rotational clutch assembly for a stent delivery catheter for delivering and deploying a radially expandable stent at a strategic orientation and location in a body vessel, said delivery catheter including an elongated shaft and a dilator device adapted for radial expansion from a non-expanded condition to a radially expanded condition, said dilator device configured to retain said stent in the non-expanded condition, said clutch assembly comprising:
a tubular transition portion having a distal end mounted to said dilator device, and a proximal portion rotatably coupled to the distal end of said elongated shaft at rotational joint for substantially free rotation about a longitudinal axis thereof relative to said elongated shaft such that said dilator device is substantially torsionally isolated from said elongated shaft, and
a pair of opposed contact elements disposed in opposed relationship to one another, one contact element being associated with the elongated shaft while the second contact element being associated with the transition portion such that during said advancement of the elongated shaft through the body vessel, the contact elements are moved into compressive mutual contact with one another to transmit axial compressive forces from the elongated shaft to the transition portion.

14. The rotational clutch assembly according to claim 13, wherein
said rotational joint includes an inwardly tapered shoulder portion coupled to one of a distal end of the elongated shaft and a proximal end of the transition portion, said rotational joint further including a neck portion extending from said tapered shoulder portion, said neck portion being formed and dimensioned for sliding rotational receipt into an opening at the other of the tubular transition portion and the elongated shaft for rotational receipt thereof.

15. The rotational clutch assembly according to claim 13, further including:
a flexible protective boot device extending circumferentially over the rotational joint having one end secured to the elongated shaft and an opposite end secured to the transition portion such that a fluid-tight seal is formed while still enabling relative rotation between the elongated shaft and the deployment device.

16. The rotational clutch assembly according to claim 13, further including:
a first support tube associated with the elongated shaft, and a second support tube associated with the transition portion, each the first and second support tube having respective end portions disposed in substantially opposed relationship to one another, each end portion supporting one of said contact elements in opposed relationship to one another.

17. The rotational clutch assembly according to claim 16, further including:
an elongated stiffening element extending substantially longitudinally across the rotational joint, one end of said stiffening element being disposed in a distal pocket defined in part by a distal end wall of the transition portion, and an opposite end of said stiffening element being disposed in a proximal pocket defined in part by a proximal end wall associated with the elongated shaft such that during said advancement of the elongated shaft through the body vessel, one end of the stiffening element contacts the distal end wall and the opposite end of the stiffening element contacts the proximal wall to transmit axial compressive forces from the elongated shaft to the transition portion.

18. The rotational clutch assembly according to claim 17, further including:
a first support tube associated with the elongated shaft and defining said distal pocket, and a second support tube associated with the transition portion and defining said proximal pocket, each the first and second support tube having respective end portions disposed in substantially opposed relationship to one another.
